(19) United States
(12) Patent Application Publication
(10) Pub. No.: US 2026/0226433 A1
(43) Pub. Date: Aug. 6, 2026

(54) REVERSE TRANSCRIPTASE MUTANTS AND USES THEREOF

(71) Applicant: Integrated DNA Technologies, Inc., Coralville, IA (US)

(72) Inventors: Sarah Franz Beaudoin, Iowa City, IA (US); Christopher Anthony Vakulskas, North Liberty, IA (US)

(21) Appl. No.: 19/429,035

(22) Filed: Dec. 22, 2025

Related U.S. Application Data
(60) Provisional application No. 63/739,262, filed on Dec. 27, 2024.

(51) Int. Cl. C12N 9/12; C12N 9/22; C12N 15/10; C12P 19/34

(52) U.S. Cl. CPC C12N 9/1276; C12N 9/226; C12N 15/1096; C12P 19/34; C07K 2319/80; C12Y 207/07049

(57) ABSTRACT

The present disclosure provides Moloney murine leukemia virus (MMLV) reverse transcriptase (RTase) variants. The present disclosure further provides amino acid positions for mutagenesis of MMLV RTase as well as nucleic acids, kits, compositions, fusion proteins, and methods including MMLV RTase variants.

### CROSS-REFERENCE TO RELATED APPLICATION(S)

This application claims the benefit of U.S. Provisional Application No. 63/739,262 filed Dec. 27, 2024 for “REVERSE TRANSCRIPTASE MUTANTS AND USES THEREOF” by S. F. Beaudoin and C. A. Vakulskas.

### FIELD OF THE INVENTION

The present disclosure relates to reverse transcriptase enzymes and, more particularly, to mutant variants of Moloney murine leukemia virus reverse transcriptase and uses thereof.

### REFERENCE TO AN ELECTRONIC SEQUENCE LISTING

The present application includes a Sequence Listing which has been submitted electronically as an XML document in the ST.26 format via Patent Center and concurrent with the filing of the application, and which is hereby incorporated by reference in its entirety. The XML document is named “2024-24213-P-US-D0487-P15916US01.xml,” was created on Dec. 11, 2024, and is 845,224 bytes in size.

### BACKGROUND

Reverses transcriptase (RTase) enzymes facilitate reverse transcription of ribonucleic acid (RNA) into copy deoxyribonucleic acid (cDNA). RTases are encoded and used by retroviruses to reverse-transcribe RNA genomic material into cDNA, which can then be integrated into the host genome. RTase enzymes are often used in laboratory applications to produce cDNA for a variety of downstream applications, including quantitative PCR, gene expression analysis, isolated RNA sequencing, gene cloning, and cDNA library creation.

### SUMMARY

An example of an isolated Moloney murine leukemia virus (MMLV) reverse transcriptase (RTase) mutant according to the present disclosure includes an isolated MMLV RTase comprising an amino acid sequence as set forth in any of SEQ ID NOs: 278-432.

An MMLV RTase variant comprising a substitution at a position corresponding to least one of position 9, position 157, position 159, position 173, position 287, position 333, position 433, position 435, position 467, position 490, position 502, position 629, or position 646 of any of SEQ ID NOs: 275-277.

An example of a nucleotide according to the present disclosure includes a nucleotide encoding an MMLV RTase disclosed herein.

An example of a cDNA according to the present disclosure includes a cDNA encoding an MMLV RTase disclosed herein.

An example of a kit according to the present disclosure includes a kit comprising an MMLV RTase disclosed herein.

An example of a composition according to the present disclosure includes a composition comprising an MMLV RTase disclosed herein.

An isolated prime editor fusion protein according to the present disclosure includes a prime editor fusion protein comprising a nuclease domain and a reverse transcriptase domain having a sequence of an MMLV RTase disclosed herein.

An example of a method of synthesizing cDNA according to the present disclosure includes providing a ribonucleic acid template and contacting the ribonucleic acid template with an MMLV RTase disclosed herein to synthesize cDNA.

An example of a method of gene editing according to the present disclosure includes providing a sample including a double-stranded target DNA sequence and contacting the double-stranded target DNA sequence with a prime editor. The prime editor includes first domain comprising a nucleic acid programmable DNA binding protein, a second domain comprising the MMLV RTase disclosed herein, and a prime editing guide RNA.

An example of a method of performing reverse transcriptase-polymerase chain reaction (RT-PCR) according to the present disclosure includes providing a ribonucleic acid template, contacting the ribonucleic acid template with an MMLV RTase of the present disclosure to synthesize cDNA, and contacting the cDNA with a DNA polymerase to amplify the cDNA.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a flow diagram of an example of a method of synthesizing cDNA.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure relates to Moloney murine leukemia virus (MMLV) reverse transcriptase (RTase) enzymes. In particular, the present disclosure provides mutant variants of MMLV RTase and sequences encoding mutant variants of MMLV RTase. The present disclosure also provides various mutagenesis primers that can be used to perform site-directed mutagenesis of an MMLV RTase nucleotide sequence to create a sequence encoding an MMLV RTase variant disclosed herein. The disclosure further provides various methods and kits that use the MMLV RTase variants disclosed herein to synthesize copy deoxyribonucleic acid (cDNA) from ribonucleic acid (RNA) template sequences.

Various RTase enzymes are used in research applications to synthesize cDNA for, e.g., polymerase chain reaction (PCR), gene-editing techniques, cDNA library construction, RNA sequencing, etc. MMLV RTase is characterized by low RNase H activity and a higher fidelity as compared to other common RTases used in laboratory applications, such as Avian Myeloblastosis Virus (AMV) RTase. The reduced RNase H activity reduces the rate at which MMLV RTase degrades RNA templates and allows MMLV RTases to be used for the reverse transcription of long RNAs (>5 kilobase) and in prime editing applications. Various MMLV RTases with further reduced RNase H activity have been constructed to provide additional reductions to RNA template degradation and, consequently, additional improvements to cDNA synthesis.

However, cDNA synthesis by MMLV RTase can be impeded by the presence of secondary structures in RNA templates and, notably, wildtype MMLV RTase has significantly reduced activity at the elevated temperatures useful for disrupting RNA. In particular, the optimal temperature for wildtype MMLV RTase activity is 37° C. However, reaction temperatures of over 45° C. or, in some examples, above 50° C. are recommended to disrupt RNA secondary structures and, further, to reduce non-specific priming (i.e., by reducing mismatch duplex stability). To improve the thermostability of MMLV RTase, various methods further attempt to substitute Mn²⁺ for Mg²⁺ as the RTase cofactor by altering reaction conditions (e.g., by performing cDNA synthesis in high Mn²⁺ conditions and in the absence of Mg²⁺) and/or by using variant MMLV RTases with enhanced affinity for Mn²⁺, but these methods generally result in increased error rates.

As will be explained in more detail subsequently, and particularly with respect to the data presented in the discussion of Examples 3-5, the MMLV RTase mutant variants disclosed herein have improved activity and thermostability as compared to wildtype MMLV RTase. Notably, the mutant variants of MMLV RTase also have improved activity and thermostability as compared to other existing mutant variants of MMLV RTase. The enhanced activity and thermostability of the MMLV RTase mutants disclosed herein is advantageous in various applications, including in PCR and prime editing applications.

One of skill in the art will recognize that cDNA molecules made using the MMLV RTase mutants of the disclosure can be used in a variety of additional downstream applications. For example, amplification methods may include one-step PCR, two-step PCR, real-time or quantitative PCR, hot-start PCR, nested PCR, touch down PCR, differential display PCR (DDRT-PCR), microarray technologies, inverse PCR, Rapid amplification of PCR ends (RACE or anchored PCR), multiplex PCR, and site directed PCR mutagenesis. Synthesized cDNA and cDNA libraries created with the MMLV RTase mutants of the disclosure can be used in cloning and/or sequencing for further characterization. One of skill in the art will recognize that nucleic acid amplification using cDNA prepared with the MMLV RTase mutants of the disclosure may include additional techniques not listed herein.

In some examples, an MMLV RTase variant according to the present disclosure can be an isolated MMLV RTase variant having a sequence of any of the MMLV RTase variants disclosed herein or a sequence that is substantially identical to any of the MMLV RTase variants disclosed herein. In yet further examples, an MMLV RTase variant according to the present disclosure can be an isolated MMLV RTase having one or more useful mutations disclosed herein.

An MMLV RTase mutant according to the present disclosure can be included in a kit for use in reverse transcription or related technologies. These kits include one or more of the following: an MMLV RTase mutant enzyme, reagents and buffers for conducting a reverse transcriptase reaction, a box, vial tubes, ampules, and the like. Kits can also include instructions for use of the kit for practicing any of the methods disclosed herein or other methods known to those of skill in the art.

Further, an MMLV RTase mutant according to the present disclosure can be a domain of a prime editor fusion protein for use in a clustered regularly interspaced short palindromic repeat (CRISPR) technique or another suitable gene-editing technique. More particularly, a fusion protein of an MMLV RTase mutant according to the present disclosure and a CRISPR-associated protein (Cas), such as a Cas9 protein (e.g., Cas9 nickase), can be constructed using any suitable technique (e.g., a suitable cloning technique). The MMLV RTase domain of the resultant fusion protein can be used to perform reverse transcription (i.e., cDNA synthesis) as part of a prime editing technique. In these examples, the MMLV RTase mutant can be included in a kit for prime editing and, optionally, can be provided with one or more prime editing guide RNAs (pegRNAs).

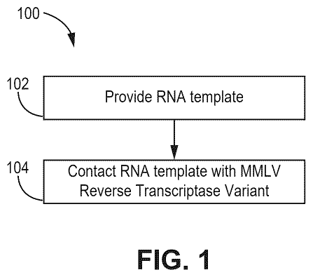

FIG. 1 is a flow diagram of method 100, which is an example of a method of synthesizing cDNA. Method 100 includes steps of 102-104 of providing a ribonucleic acid template (step 102) and contacting the ribonucleic acid template with an MMLV RTase variant (104). Method 100 can be performed as part of a PCR method, a prime editing method, or any other suitable method using a reverse transcriptase to produce a cDNA.

In step 102, a ribonucleic acid template is provided. The template can be provided by any suitable method and, in some examples, can be added to a reaction mixture including one or more buffer components, deoxynucleotide triphosphates (dNTPs), and/or any other suitable components.

In step 104, the ribonucleic acid template provided in step 120 is contacted with an MMLV RTase variant. Contacting ribonucleic acid template with the MMLV RTase variant allows the MMLV RTase variant to reverse transcribe the ribonucleic acid template to produce a cDNA.

Where method 100 is used to perform a PCR technique, the template provided in step 102 can be provided, for example, as part of a reaction mixture including one or more buffer components, deoxynucleotide triphosphates (dNTPs), etc. In step 104, the MMLV RTase variant can be added to the reaction mixture, thereby allowing the MMLV RTase variant to contact the template and synthesize cDNA. The cDNA can subsequently be amplified in a separate reaction (i.e., as part of a two-step procedure) or in the same reaction (i.e., as part of a one-step procedure) using a DNA polymerase. Amplified cDNA product can, in some use cases, be quantified, analyzed, etc. using any suitable technique (e.g., a qPCR technique).

Where method 100 is used to perform a prime editing technique, the ribonucleic acid template provided in step 102 can be a template included in a pegRNA. The pegRNA can include a protospacer element for targeting a particular genomic region for gene-editing. In step 104, nuclease domain of the prime editor fusion protein (e.g., a Cas9 nickase domain) can introduce a single-strand cut to allow a primer-binding sequence (PBS) to anneal to an end of the nicked strand. The PBS can be adjacent or substantially to the RNA template in the pegRNA and is complementary to the free 3′ end of the nicked strand, allowing the free 3′ end to be used as a primer for cDNA synthesis. After the PBS anneals to the end of the nicked strand, the variant MMLV RTase domain of the prime editor fusion protein then contacts the RNA template to perform cDNA synthesis. The resultant heteroduplex can then be resolved via a mismatch repair mechanism.

### EXAMPLES

The following examples are illustrative and are not intended to limit the scope of the invention.

### Example 1—Preparation of MMLV RTase Mutants by Site Directed Mutagenesis

The MMLV RTase variants described herein were prepared by introducing various mutations to a base construct that encodes a variant of MMLV RTase having thirteen mutations (SEQ ID NO: 119, encoding SEQ ID NO: 277) relative to the wildtype sequence (SEQ ID NO: 117, encoding SEQ ID NO: 275). The base construct having the amino acid sequence set forth in SEQ ID NO: 277 (and encoded by the DNA sequence set forth in SEQ ID NO: 119) is referred to herein as the “M63 variant” and the “M63 MMLV RTase.” The M63 variant is a variant of wild-type MMLV RTase (SEQ ID NO: 117, encoding SEQ ID NO: 275) including three mutations contained within SuperScript II (SSII) RTase (Invitrogen; SEQ ID NO: 118, encoding SEQ ID NO: 276) and ten additional mutations identified in U.S. Patent Publication No. 2023/0031558. The three mutations of the SuperScript II RTase (SEQ ID NO: 276) have been shown to reduce RNase activity. Further, the additional ten mutations of the M63 variant (SEQ ID NO: 275) have been shown to improve activity and thermostability as compared to wild-type MMLV RTase (U.S. Pat. Pub. No. 2023/0031558).

The mutations contained by the variants disclosed herein (SEQ IDs: 278-432) were obtained by site-directed mutagenesis of the M63 variant using standard PCR conditions and relevant primers. Primers used for construction of each mutation are provided in Table 1. The resultant plasmids were transformed into E. coli BL21 (DE3) for expression. The M63 variant base construct was contained in a pET28a vector and all variant amino acid sequences contain an N-terminal 20mer 6× His-tag having a sequence of “MGSSHHHHHHSSGLVPRGSH.” However, for clarity and convenience, mutation positions herein are referred to by their position relative to the methionine following the N-terminal 20mer encoded by the pET28a vector.

The oligonucleotide primers in Table 1 include “TOP” or “BTM” designations, which refer to top and bottom primers used for site-directed mutagenesis. Table 1 includes 58 pairs of oligonucleotide primers for creating 58 mutations to the MMLV RTase M63 variant. Each pair has the same “Primer Name,” except for the “TOP” or “BTM” designations, and SEQ IDs 1-116 are ordered such that SEQ IDs 1-58 are paired to respective ones of SEQ IDs 59-116. One pair of corresponding primers (e.g., SEQ ID 1 and SEQ ID 59, SEQ ID 2 and SEQ ID 60, etc.) were used to introduce each mutation via site-directed mutagenesis. In the examples in Table 1, each pair of corresponding primers are reverse complements. Although all MMLV variants disclosed herein were obtained by site-directed mutagenesis of the M63 MMLV RTase sequence (SEQ ID NO: 119), for clarity and convenience, mutations in MMLV RTase sequences are identified using amino acid identities of the wildtype sequence.

TABLE 1
Sequences of oligonucleotide primers used for site-directed mutagenesis of the M63 variant
of MMLV RTase.

| SEQ | | |
|---|---|---|
| ID | Primer Name | Primer Sequence (5′-3′) |
| 1 | MMLV_M63 | GCAGCCATATGACTTTAAATATTGAGGATCCCCATCGTTTACATGAGACATCAAAAGAAC |
| | E7P TOP | |
| 2 | MMLV_M63 | CCATATGACTTTAAATATTGAGGATGAGCATTTATTACATGAGACATCAAAAGAACCCGAC |
| | R9L TOP | |
| 3 | MMLV_M63 | CCATATGACTTTAAATATTGAGGATGAGCATGCGTTACATGAGACATCAAAAGAACCCGAC |
| | R9A TOP | |
| 4 | MMLV_M63 | GCGTAGAAGACATCCATCCGACTTATCCTAATCCTTATAATCTGTTATCAGGCCT |
| | V129Y TOP | |
| 5 | MMLV_M63 | CCGACTGTACCTAATCCTTATAATCTGGCGTCAGGCCTGCCCCCATCGC |
| | L136A TOP | |
| 6 | MMLV_M63 | CGACTGTACCTAATCCTTATAATCTGTTATTCGGCCTGCCCCCATCGC |
| | S137F TOP | |
| 7 | MMLV_M63 | GACTGTACCTAATCCTTATAATCTGTTATCATCTCTGCCCCCATCGCACCAAT |
| | G138S TOP | |
| 8 | MMLV_M63 | CAGTATTAGACTTGAAAGACGCGTTCGTATGCCTGCGTCTGCACCCAA |
| | F156V TOP | |
| 9 | MMLV_M63 | GTATTAGACTTGAAAGACGCGTTCTTTGCGCTGCGTCTGCACCCAACGT |
| | C157A TOP | |
| 10 | MMLV_M63 | CCGCTGTTTGCGTTCGAATGGGAGGATCCTGAAATGGGAATTTCGGGT |
| | R173E TOP | |
| 11 | MMLV_M63 | GCCCCAGGGCTTTAAAAACAGCGCGACATTGTTCGATGAAGCACTTCACCGT |
| | P196A TOP | |
| 12 | MMLV_M63 | GCCCCAGGGCTTTAAAAACAGCTTAACATTGTTCGATGAAGCACTTCACCGT |
| | P196L TOP | |
| 13 | MMLV_M63 | GCCCCAGGGCTTTAAAAACAGCCATACATTGTTCGATGAAGCACTTCACCGT |
| | P196H TOP | |
| 14 | MMLV_M63 | TAACCAAGCCCTTTGAGCTGTTCTCTGATGAAAAACAGGGATATGCAAAAGGA |
| | V370S TOP | |
| 15 | MMLV_M63 | CCCTTTGAGCTGTTCGTTGATGAAAAATTAGGATATGCAAAAGGAGTATTAACCCAA |
| | Q374L TOP | |
| 16 | MMLV_M63 | GCTGTTCGTTGATGAAAAACAGGGAATTGCAAAAGGAGTATTAACCCAAAAGTTAGGCCCGT |
| | Y376I TOP | |
| 17 | MMLV_M63 | GATGAAAAACAGGGATATGCAAAAGGATCTTTAACCCAAAAGTTAGGCCCGTGGCGT |
| | V380S TOP | |
| 18 | MMLV_M63 | CCTGTTGCTTACTTGAGTAAAAAATTGGATTTAGTCGCAGCAGGATGGCCACCGT |
| | P401L TOP | |
| 19 | MMLV_M63 | CAATTGCCGTTTTGACAAAGGATGCATCTAAGTTGACGATGGGTCAACCCTT |
| | G424S TOP | |
| 20 | MMLV_M63 | GTTTTGACAAAGGATGCAGGTAAGTTGTGTATGGGTCAACCCTTAAACATCTTGGCT |
| | T427C TOP | |
| 21 | MMLV_M63 | AGTTGACGATGGGTCAACCCTTAACAATCTTGGCTCCACATGCTGTAGA |
| | V433T TOP | |
| 22 | MMLV_M63 | CGATGGGTCAACCCTTAAACATCCATGCTCCACATGCTGTAGAAGCGT |
| | K435H TOP | |
| 23 | MMLV_M63 | GATGGGTCAACCCTTAAACATCTTGTCTCCACATGCTGTAGAAGCGTTAGT |
| | A436S TOP | |
| 24 | MMLV_M63 | ACTATCAGGCGCTTCTGCTTGATAACGATCGTGTACAATTTGGACCAGTTGT |
| | T467N TOP | |
| 25 | MMLV_M63 | CTTGATACGGATCGTGTACAATTTGGATCTGTTGTAGCTTTGAATCCAGCTACTTTGC |
| | P474S TOP | |
| 26 | MMLV_M63 | GATCGTGTACAATTTGGACCAGTTGTATATTTGAATCCAGCTACTTTGCTTCCCCT |
| | A477Y TOP | |
| 27 | MMLV_M63 | GCTTCCCCTTCCAGAAGAAGGAACACAGCACAATTGTTTAGATATTCTGGCCGA |
| | LA91T TOP | |
| 28 | MMLV_M63 | GACTTCAGCACAATTGTTTAGATATTCTGCAAGAGGCACATGGGACGCGCCCT |
| | A500Q TOP | |
| 29 | MMLV_M63 | CAGCACAATTGTTTAGATATTCTGGCCGAGTAACAAAGCCCGAAAGGAAGCTGAG |
| | A502STOP | |
| | TOP | |
| 30 | MMLV_M63 | GTTTAGATATTCTGGCCGAGGCACGTGGGACGCGCCCTGATTTGA |
| | H503R TOP | |
| 31 | MMLV_M63 | TAGATATTCTGGCCGAGGCACATGCGACGCGCCCTGATTTGACGGA |
| | G504A TOP | |
| 32 | MMLV_M63 | CTGGCCGAGGCACATGGGTCTCGCCCTGATTTGACGGATCAGC |
| | T505S TOP | |
| 33 | MMLV_M63 | CTGATGCCGACCATACATGGTATTTAGGCGGCAGTAGTCTTCTTCAAGA |
| | T523L TOP | |
| 34 | MMLV_M63 | GATGCCGACCATACATGGTATACTAACGGCAGTAGTCTTCTTCAAGAGGGGCA |
| | G524N TOP | |
| 35 | MMLV_M63 | CATGGTATACTGGCGGCAGTAGTGTACTTCAAGAGGGGCAACGCAAGGCGGGA |
| | L528V TOP | |
| 36 | MMLV_M63 | CATGGTATACTGGCGGCAGTAGTTATCTTCAAGAGGGGCAACGCAAGGCGGGA |
| | L528Y TOP | |
| 37 | MMLV_M63 | ATACTGGCGGCAGTAGTCTTCTTGTAGAGGGGCAACGCAAGGCGGGA |
| | Q530V TOP | |
| 38 | MMLV_M63 | CTGGCGGCAGTAGTCTTCTTCAATCTGGGCAACGCAAGGCGGGA |
| | E531S TOP | |
| 39 | MMLV_M63 | CTTCAAGAGGGGCAACGCAAGTAACAAAGCCCGAAAGGAAGCTGAG |
| | A536STOP | |
| | TOP | |
| 40 | MMLV_M63 | GAGACCGAAGTTATCTGGGCCTGGGCGTTACCCGCGGGAACAT |
| | K550W TOP | |
| 41 | MMLV_M63 | TTGAATGTCTACACCAACTCACGTTATACATTTGCAACAGCGCATTGGCATGGCGA |
| | A587T TOP | |
| 42 | MMLV_M63 | CGTTATGCTTTTGCAACAGCGCATTTACATGGCGAAATTTACCGCCGCCGT |
| | W593L TOP | |
| 43 | MMLV_M63 | CCGCCGCCGTGGTCTGGGGACTAGTGAGGGTAAGGAAATTAAAAATAAAGA |
| | L604G TOP | |
| 44 | MMLV_M63 | GCCGTGGTCTGCTGACTAGTTCTGGTAAGGAAATTAAAAATAAAGATGAGATTCTTGC |
| | E607S TOP | |
| 45 | MMLV_M63 | GACTAGTGAGGGTAAGGAAATTAAAAATAAACCCGAGATTCTTGCGTTGTTAAAAGCTTTATTC |
| | D615P TOP | |
| 46 | MMLV_M63 | GACTAGTGAGGGTAAGGAAATTAAAAATAAACGTGAGATTCTTGCGTTGTTAAAAGCTTTATTC |
| | D615R TOP | |
| 47 | MMLV_M63 | TGAGGGTAAGGAAATTAAAAATAAAGATGAGGTACTTGCGTTGTTAAAAGCTTTATTCTTACC |
| | I617V TOP | |
| 48 | MMLV_M63 | GGGTAAGGAAATTAAAAATAAAGATGAGATTCTTTTATTGTTAAAAGCTTTATTCTTACCAAAACGCCT |
| | A619L TOP | |
| 49 | MMLV_M63 | GGGTAAGGAAATTAAAAATAAAGATGAGATTCTTCGTTTGTTAAAAGCTTTATTCTTACCAAAACGCCT |
| | A619R TOP | |
| 50 | MMLV_M63 | TGCGTTGTTAAAAGCTTTATTCTTACCAAAAACACTTTCGATCATTCATTGCCCGGGGCA |
| | R629T TOP | |
| 51 | MMLV_M63 | ACCCCTTAACAAAAACAGGGACGTCTTTCAACTGGGGGCCAGACC |
| | L333S TOP | |
| 52 | MMLV_M63 | ACCCCTTAACAAAAACAGGGACGGAGTTCAACTGGGGGCCAGACC |
| | L333E TOP | |
| 53 | MMLV_M63 | CTTTGCTTCCCCTTCCAGAAGAAGCGCTTCAGCACAATTGTTTAGATATTCTGGCCGA |
| | G490A TOP | |
| 54 | MMLV_M63 | CTTTGCTTCCCCTTCCAGAAGAAAACCTTCAGCACAATTGTTTAGATATTCTGGCCGA |
| | G490N TOP | |
| 55 | MMLV_M63 | CAAAAGGGTCACTCAGCGGAGGTACGTGGAAACCGTATGGCGGA |
| | A646V TOP | |
| 56 | MMLV_M63 | GGTATTTGCTGAAAGAAGGTCAACGTCGTATTACTGATGCGCGTAAGGAGAC |
| | W279R TOP | |
| 57 | MMLV_M63 | TTGGATTACTGATGCGCGTAAGGAGGATGTAATGGGGCAGCCTACGCC |
| | T287D TOP | |
| 58 | MMLV_M63 | CGTTATGCTTTTGCAACAGCGCATTTACATGGCGAAATTTACCGCCGCCGT |
| | W593L TOP | |
| 59 | MMLV_M63 | GTTCTTTTGATGTCTCATGTAAACGATGGGGATCCTCAATATTTAAAGTCATATGGCTGC |
| | E7P BTM | |
| 60 | MMLV_M63 | GTCGGGTTCTTTTGATGTCTCATGTAATAAATGCTCATCCTCAATATTTAAAGTCATATGG |
| | R9L BTM | |
| 61 | MMLV_M63 | GTCGGGTTCTTTTGATGTCTCATGTAACGCATGCTCATCCTCAATATTTAAAGTCATATGG |
| | R9A BTM | |
| 62 | MMLV_M63 | AGGCCTGATAACAGATTATAAGGATTAGGATAAGTCGGATGGATGTCTTCTACGC |
| | V129Y BTM | |
| 63 | MMLV_M63 | GCGATGGGGGCAGGCCTGACGCCAGATTATAAGGATTAGGTACAGTCGG |
| | L136A BTM | |
| 64 | MMLV_M63 | GCGATGGGGGCAGGCCGAATAACAGATTATAAGGATTAGGTACAGTCG |
| | S137F BTM | |
| 65 | MMLV_M63 | ATTGGTGCGATGGGGGCAGAGATGATAACAGATTATAAGGATTAGGTACAGTC |
| | G138S BTM | |
| 66 | MMLV_M63 | TTGGGTGCAGACGCAGGCATACGAACGCGTCTTTCAAGTCTAATACTG |
| | F156V BTM | |
| 67 | MMLV_M63 | ACGTTGGGTGCAGACGCAGCGCAAAGAACGCGTCTTTCAAGTCTAATAC |
| | C157A BTM | |
| 68 | MMLV_M63 | ACCCGAAATTCCCATTTCAGGATCCTCCCATTCGAACGCAAACAGCGG |
| | R173E BTM | |
| 69 | MMLV_M63 | ACGGTGAAGTGCTTCATCGAACAATGTCGCGCTGTTTTTAAAGCCCTGGGGC |
| | P196A BTM | |
| 70 | MMLV_M63 | ACGGTGAAGTGCTTCATCGAACAATGTTAAGCTGTTTTTAAAGCCCTGGGGC |
| | P196L BTM | |
| 71 | MMLV_M63 | ACGGTGAAGTGCTTCATCGAACAATGTATGGCTGTTTTTAAAGCCCTGGGGC |
| | P196H BTM | |
| 72 | MMLV_M63 | TCCTTTTGCATATCCCTGTTTTTCATCAGAGAACAGCTCAAAGGGCTTGGTTA |
| | V370S BTM | |
| 73 | MMLV_M63 | TTGGGTTAATACTCCTTTTGCATATCCTAATTTTTCATCAACGAACAGCTCAAAGGG |
| | Q374L BTM | |
| 74 | MMLV_M63 | ACGGGCCTAACTTTTGGGTTAATACTCCTTTTGCAATTCCCTGTTTTTCATCAACGAACAGC |
| | Y376I BTM | |
| 75 | MMLV_M63 | ACGCCACGGGCCTAACTTTTGGGTTAAAGATCCTTTTGCATATCCCTGTTTTTCATC |
| | V380S BTM | |
| 76 | MMLV_M63 | ACGGTGGCCATCCTGCTGCGACTAAATCCAATTTTTTACTCAAGTAAGCAACAGG |
| | P401L BTM | |
| 77 | MMLV_M63 | AAGGGTTGACCCATCGTCAACTTAGATGCATCCTTTGTCAAAACGGCAATTG |
| | G424S BTM | |
| 78 | MMLV_M63 | AGCCAAGATGTTTAAGGGTTGACCCATACACAACTTACCTGCATCCTTTGTCAAAAC |
| | T427C BTM | |
| 79 | MMLV_M63 | TCTACAGCATGTGGAGCCAAGATTGTTAAGGGTTGACCCATCGTCAACT |
| | V433T BTM | |
| 80 | MMLV_M63 | ACGCTTCTACAGCATGTGGAGCATGGATGTTTAAGGGTTGACCCATCG |
| | K435H BTM | |
| 81 | MMLV_M63 | ACTAACGCTTCTACAGCATGTGGAGACAAGATGTTTAAGGGTTGACCCATC |
| | A436S BTM | |
| 82 | MMLV_M63 | ACAACTGGTCCAAATTGTACACGATCGTTATCAAGCAGAAGCGCCTGATAGT |
| | T467N BTM | |
| 83 | MMLV_M63 | GCAAAGTAGCTGGATTCAAAGCTACAACAGATCCAAATTGTACACGATCCGTATCAAG |
| | P474S BTM | |
| 84 | MMLV_M63 | AGGGGAAGCAAAGTAGCTGGATTCAAATATACAACTGGTCCAAATTGTACACGATC |
| | A477Y BTM | |
| 85 | MMLV_M63 | TCGGCCAGAATATCTAAACAATTGTGCTGTGTTCCTTCTTCTGGAAGGGGAAGC |
| | L491T BTM | |
| 86 | MMLV_M63 | AGGGCGCGTCCCATGTGCCTCTTGCAGAATATCTAAACAATTGTGCTGAAGTC |
| | A500Q BTM | |
| 87 | MMLV_M63 | CTCAGCTTCCTTTCGGGCTTTGTTACTCGGCCAGAATATCTAAACAATTGTGCTG |
| | A502STOP | |
| | BTM | |
| 88 | MMLV_M63 | TCAAATCAGGGCGCGTCCCACGTGCCTCGGCCAGAATATCTAAAC |
| | H503R BTM | |
| 89 | MMLV_M63 | TCCGTCAAATCAGGGCGCGTCGCATGTGCCTCGGCCAGAATATCTA |
| | G504A BTM | |
| 90 | MMLV_M63 | GCTGATCCGTCAAATCAGGGCGAGACCCATGTGCCTCGGCCAG |
| | T505S BTM | |
| 91 | MMLV_M63 | TCTTGAAGAAGACTACTGCCGCCTAAATACCATGTATGGTCGGCATCAG |
| | T523L BTM | |
| 92 | MMLV_M63 | TGCCCCTCTTGAAGAAGACTACTGCCGTTAGTATACCATGTATGGTCGGCATC |
| | G524N BTM | |
| 93 | MMLV_M63 | TCCCGCCTTGCGTTGCCCCTCTTGAAGTACACTACTGCCGCCAGTATACCATG |
| | L528V BTM | |
| 94 | MMLV_M63 | TCCCGCCTTGCGTTGCCCCTCTTGAAGATAACTACTGCCGCCAGTATACCATG |
| | L528Y BTM | |
| 95 | MMLV_M63 | TCCCGCCTTGCGTTGCCCCTCTACAAGAAGACTACTGCCGCCAGTAT |
| | Q530V BTM | |
| 96 | MMLV_M63 | TCCCGCCTTGCGTTGCCCAGATTGAAGAAGACTACTGCCGCCAG |
| | E531S BTM | |
| 97 | MMLV_M63 | CTCAGCTTCCTTTCGGGCTTTGTTACTTGCGTTGCCCCTCTTGAAG |
| | A536STOP | |
| | BTM | |
| 98 | MMLV_M63 | ATGTTCCCGCGGGTAACGCCCAGGCCCAGATAACTTCGGTCTC |
| | K550W | |
| | BTM | |
| 99 | MMLV_M63 | TCGCCATGCCAATGCGCTGTTGCAAATGTATAACGTGAGTTGGTGTAGACATTCAA |
| | A587T BTM | |
| 100 | MMLV_M63 | ACGGCGGCGGTAAATTTCGCCATGTAAATGCGCTGTTGCAAAAGCATAACG |
| | W593L BTM | |
| 101 | MMLV_M63 | TCTTTATTTTTAATTTCCTTACCCTCACTAGTCCCCAGACCACGGCGGCGG |
| | L604G BTM | |
| 102 | MMLV_M63 | GCAAGAATCTCATCTTTATTTTTAATTTCCTTACCAGAACTAGTCAGCAGACCACGGC |
| | E607S BTM | |
| 103 | MMLV_M63 | GAATAAAGCTTTTAACAACGCAAGAATCTCGGGTTTATTTTTAATTTCCTTACCCTCACTAGTC |
| | D615P BTM | |
| 104 | MMLV_M63 | GAATAAAGCTTTTAACAACGCAAGAATCTCACGTTTATTTTTAATTTCCTTACCCTCACTAGTC |
| | D615R BTM | |
| 105 | MMLV_M63 | GGTAAGAATAAAGCTTTTAACAACGCAAGTACCTCATCTTTATTTTTAATTTCCTTACCCTCA |
| | I617V BTM | |
| 106 | MMLV_M63 | AGGCGTTTTGGTAAGAATAAAGCTTTTAACAATAAAAGAATCTCATCTTTATTTTTAATTTCCTTACCC |
| | A619L BTM | |
| 107 | MMLV_M63 | AGGCGTTTTGGTAAGAATAAAGCTTTTAACAAACGAAGAATCTCATCTTTATTTTTAATTTCCTTACCC |
| | A619R BTM | |
| 108 | MMLV_M63 | TGCCCCGGGCAATGAATGATCGAAAGTGTTTTTGGTAAGAATAAAGCTTTTAACAACGCA |
| | R629T BTM | |
| 109 | MMLV_M63 | GGTCTGGCCCCCAGTTGAAAGACGTCCCTGTTTTTGTTAAGGGGT |
| | L333S BTM | |
| 110 | MMLV_M63 | GGTCTGGCCCCCAGTTGAACTCCGTCCCTGTTTTTGTTAAGGGGT |
| | L333E BTM | |
| 111 | MMLV_M63 | TCGGCCAGAATATCTAAACAATTGTGCTGAAGCGCTTCTTCTGGAAGGGGAAGCAAAG |
| | G490A SDM | |
| | BTM | |
| 112 | MMLV_M63 | TCGGCCAGAATATCTAAACAATTGTGCTGAAGGTTTTCTTCTGGAAGGGGAAGCAAAG |
| | G490N SDM | |
| | BTM | |
| 113 | MMLV_M63 | TCCGCCATACGGTTTCCACGTACCTCCGCTGAGTGACCCTTTTG |
| | A646V SDM | |
| | BTM | |
| 114 | MMLV_M63 | GTCTCCTTACGCGCATCAGTAATACGACGTTGACCTTCTTTCAGCAAATACC |
| | W279R | |
| | BTM | |
| 115 | MMLV_M63 | GGCGTAGGCTGCCCCATTACATCCTCCTTACGCGCATCAGTAATCCAA |
| | T287D BTM | |
| 116 | MMLV_M63 | ACGGCGGCGGTAAATTTCGCCATGTAAATGCGCTGTTGCAAAAGCATAACG |
| | W593L BTM | |

### Example 2—Preparation of Reverse Transcriptase Mutants for Evaluation of Activity and Thermostability

The data presented in Tables 2-14 was generated by in vitro assay of purified recombinant protein. All MMLV variants were generated in substantially the same manner and as is outlined herein.

A colony with the appropriate strain was used to inoculate 1 mL of TB media in a 96-well deep well plate. The culture was provided with kanamycin at a concentration of 0.05 mg/mL and grown at 37° C. When the culture reached an OD of approximately 0.8, the plate was cooled on ice for 5 minutes. A 5 μL volume of 100 mM IPTG was added to each culture to induce protein expression, followed by incubation at 18° C. for 23 hours. Following induction, cells were harvested by centrifugation at 4,700 rpm for 10 minutes to produce a cell pellet. Cell pellets were produced in the aforementioned example for each examined MMLV RTase variant.

Each resultant cell pellet was resuspended in a lysis buffer containing 50 mM NaPO₄, 5% glycerol, 300 mM NaCl, and 10 mM imidazole. Resuspended cells were lysed by the addition of 1× BugBuster® (Millipore Sigma, Cat #70921) and incubation on an end-over-end mixer for 15 minutes at room temperature. Each lysate was subsequently centrifuged at 4,700 rpm for 10 minutes and at a temperature of 4° C. to remove cell debris.

The cleared lysates were applied to a HisPur™ Ni-NTA spin plate (ThermoFisher, Cat #88230). The resin was equilibrated with Screening His-Bind buffer (50 mM NaPO₄, pH 7.8, 5% glycerol, 300 mM NaCl, and 10 mM imidazole) and cleared lysate was subsequently loaded. Samples were washed three times with Screening His-Wash buffer (50 mM NaPO₄, pH 7.8, 5% glycerol, 300 mM NaCl, and 25 mM imidazole) and eluted using Screening His-Elution buffer (50 mM NaPO₄, pH 7.8, 5% glycerol, 300 mM NaCl, and 250 mM imidazole). Purified proteins were normalized to a set concentration of 130 mM for testing purposes.

The ability of each variant of the M63 variant base construct to synthesize cDNA from purified total Universal RNA (Agilent) was compared to purified protein having the sequence of SSII (Invitrogen; SEQ ID NO: 276) and the M63 variant base construct. In particular, MMLV RTase activity was assayed by random hexamer priming using a standard two-step cDNA synthesis. Thermostability was assessed by varying the assay temperature. Protein having the sequence of SSII (Invitrogen) was prepared according to the method given in Example 2 using BL21 (DE3) transformed with a plasmid containing SEQ ID NO: 118, and is referred to herein as “MMLV-II.”

### Example 3—Evaluation of MMLV Reverse Transcriptase Single Mutants

RTases (2 μL 130 nM or 13 nM) were added to a reaction mixture containing RNA (50 ng), dNTPs (100 μM), random hexamer primers (5 ng/μL), first strand synthesis buffer (1×, 50 mM Tris-HCl, pH 8.3, 75 mM KCl, 3 mM MgCl₂, 10 mM DTT), and SuperaseIN (ThermoFisher; 0.17 U/μL) in a 50 μL volume. The reaction was allowed to proceed at a target temperature for a duration of 15 minutes, followed by incubation at 80° C. for 10 minutes. Multiple iterations of the two-step protocol were performed to vary the target temperature. As will be discussed in more detail subsequent, reverse transcription by random hexamer priming was performed at 50° C., 55° C., 60° C., or 65° C.

Cycle threshold (Ct) values describing the cDNA synthesis activity of the RTase mutants were quantified by qPCR amplification using an assay that identified the hypoxanthine phosphoribosyltransferase (HPRT) gene or the serine and arginine rich splicing factor 9 (SFRS9) gene in human cells. Both HPRT and SFRS9 were assayed for each MMLV RTase variant. An assay master mix including Integrated DNA Technologies PrimeTime® Gene Expression Master Mix (GEM) was prepared and used to prepare each assay. The master mix also included primers and probes for HPRT and SFRS9, for which sequence information is presented in Table 2. In the sequences listed in the “Primer/Probe Sequence (5′-3′)” column, “5SUN” refers to a 5′ SUN (Integrated DNA Technologies) modification, “ZEN” refers to the ZEN internal quencher (Integrated DNA Technologies), “3IABKFQ” refers to 3′ Iowa Black® FQ (Integrated DNA Technologies) modification, and “56-FAM” refers to a 5′ 6-FAM (Fluorescein; Integrated DNA Technologies) modification. In particular, the assay master mix included GEM (1×), HPRT and SFRS9 primer sets (500 nM each, Table 2), and HPRT and SFRS9 probes (250 nM each, Table 2).

The assay master mix and synthesized cDNA (i.e., from the prior reaction) were mixed at a 10:1 ratio for a final volume of 20 μL. qPCR reactions proceeded on a QuantStudio7 Flex qPCR machine (ThermoFisher) for 40 cycles using the following cycle conditions: 95° C. hold for 3 minutes, 95° C. for 15 seconds, and 60° C. for one minute.

TABLE 2
Sequences of primers and probes used for qPCR assays.

| | Primer | |
|---|---|---|
| Seq ID | Name | Primer/Probe Sequence (5′-3′) |
| 433 | Hs SFRS9 | GTCGAGTATCTCAGAAAAGAAGACA |
| | Forward | |
| | Primer | |
| 434 | Hs SFRS9 | CTCGGATGTAGGAAGTTTCACC |
| | Reverse | |
| | Primer | |
| 435 | Hs SFRS9 | /5SUN/ATGCCCTGC/ZEN/GTAAACTGGATGACA/3IABkFQ/ |
| | Probe- | |
| | SUN | |
| 436 | Hs HPRT | GACTTTGCTTTCCTTGGTCAG |
| | Forward | |
| | Primer | |
| 437 | Hs HPRT | GGCTTATATCCAACACTTCGTG |
| | Reverse | |
| | Primer | |
| 438 | Hs HPRT | /56- |
| | Probe- | FAM/ATGGTCAAG/ZEN/GTCGCAAGCTTGCTGGT/3IABkFQ/ |
| | FAM | |

A646V, G490A, L333E, K435H, R159P, R159S, R629M and T287D mutants were generated by site directed mutagenesis according to the method described subsequently with respect to Example 1. The amino acid sequences of the above-indicated mutants as well as MMLV-II and the M63 variant MMLV RTase are provided in Table 3, below.

TABLE 3

| MMLV_RTase | | |
|---|---|---|
| Variant Name | SEQ ID | Amino Acid Sequence (N-C) |
| A646V | 432 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEVRGNRMADQA |
| | | ARKAAITETPDTSTLLIENSSPYTSEHF |
| G490A | 430 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEALQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| L333E | 428 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| K435H | 429 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| R159P | 425 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| R159S | 426 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| R629M | 431 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKMLSIIHCPGHQKGHSAEARGNRMADQ |
| | | AARKAAITETPDTSTLLIENSSPYTSEHF |
| T287D | 427 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 Variant | 277 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| MMLV-II | 276 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSQEARLGIKPHIQRLLDQGILVPCQSPW |
| | | NTPLLPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWLT |
| | | EARKETVMGQPTPKTPRQLREFLGTAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLVILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHIHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |

The two-step reactions for the MMLV variants were analyzed and reported by Ct value. Table 4, Table 5, Table 6, and Table 7 report Ct values for 50° C., 55° C., 60° C., and 65° C., respectively, with an RTase concentration of 5.2 nM. Table 8 reports Ct values at 65° C. with an RTase concentration of 0.52 nM.

All single mutants provided in Table 3 (SEQ IDs: 425-432) showed increased activity relative to both the M63 variant and MMLV-II at 50° C., 55° C., 60° C., and 65° C. (at 5.2 nM RTase) for both assay targets (i.e., HPRT and SFRS9). The A646V, G490A, L333E, K435, R159P, R159S, and T287D mutants also showed increased activity at 65° C. by the assay for SFRS9, while the R629M mutant showed increased activity relative to MMLV-II and comparable activity relative to the M63 variant. At 65° C. and using the lower enzyme RTase concentration the A646V, G490A, R159P, R159S, T287D mutants showed increased activity relative to both the M63 variant and MMLV-II for both gene assays. At the lower concentration of RTase (i.e., 0.52 nM) MMLV-II also showed increased activity relative to the M63 variant according to the assay for SFRS9. Moreover, the M63 variant also includes a mutation at position 435 (K435L). Notably, the K435H mutant shows increased activity relative to the M63 variant at 50° C., 55° C., 60° C., and 65° C.

TABLE 4
Two-Step cDNA synthesis by mutants of modified MMLV
RTase. Data was generated via qPCR assay for HPRT OR
SFRS9, as indicated by the “Target Name” column. Data is
reported as Ct values. Reaction temperatures were 50° C. and
the indicated MMLV RTase variant was
at a concentration of 5.2 nM.

| | Target | Ct | Ct |
|---|---|---|---|
| MMLV RTase Variant | Name | Mean | SD |
| A646V | HPRT | 29.32 | 0.04 |
| | SFRS9 | 28.54 | 0.02 |
| G490A | HPRT | 29.74 | 0.07 |
| | SFRS9 | 28.95 | 0.06 |
| L333E | HPRT | 29.18 | 0.05 |
| | SFRS9 | 28.43 | 0.03 |
| K435H | HPRT | 29.33 | 0.36 |
| | SFRS9 | 28.63 | 0.30 |
| R159P | HPRT | 29.37 | 0.07 |
| | SFRS9 | 28.75 | 0.05 |
| R159S | HPRT | 29.77 | 0.03 |
| | SFRS9 | 29.03 | 0.05 |
| R629M | HPRT | 29.85 | 0.03 |
| | SFRS9 | 29.08 | 0.01 |
| T287D | HPRT | 28.97 | 0.06 |
| | SFRS9 | 28.27 | 0.03 |
| M63 Variant | HPRT | 30.02 | 0.13 |
| | SFRS9 | 29.33 | 0.08 |
| MMLV-II | HPRT | 39.48 | 0.73 |
| | SFRS9 | 37.84 | 0.56 |

TABLE 5
Two-Step cDNA synthesis by triple and quadruple
mutants of modified MMLV RTase. Data was generated
via qPCR assay for HPRT OR SFRS9, as indicated by the
“Target Name” column. Data is reported as Ct values.
Reaction temperatures were 55° C. and the indicated
MMLV RTase variant was at a concentration of 5.2 nM.

| | Target | Ct | Ct |
|---|---|---|---|
| RTase | Name | Mean | SD |
| A646V | HPRT | 29.627 | 0.057 |
| | SFRS9 | 28.983 | 0.011 |
| G490A | HPRT | 30.208 | 0.113 |
| | SFRS9 | 29.497 | 0.062 |
| L333E | HPRT | 29.583 | 0.064 |
| | SFRS9 | 28.999 | 0.023 |
| K435H | HPRT | 29.985 | 0.334 |
| | SFRS9 | 29.369 | 0.349 |
| R159P | HPRT | 30.109 | 0.064 |
| | SFRS9 | 29.544 | 0.029 |
| R159S | HPRT | 30.330 | 0.053 |
| | SFRS9 | 29.565 | 0.052 |
| R629M | HPRT | 30.412 | 0.021 |
| | SFRS9 | 29.795 | 0.080 |
| T287D | HPRT | 29.715 | 0.025 |
| | SFRS9 | 29.156 | 0.080 |
| M63 Variant | HPRT | 30.785 | 0.061 |
| | SFRS9 | 30.224 | 0.047 |
| MMLV-II | HPRT | 39.638 | 0.513 |
| | SFRS9 | 38.546 | 0.533 |

TABLE 6
Two-Step cDNA synthesis by triple and quadruple
mutants of modified MMLV RTase. Data was generated
via qPCR assay for HPRT OR SFRS9, as indicated by
the “Target Name” column. Data is reported as Ct
values. Reaction temperatures were 60° C. and the
indicated MMLV RTase variant was at a concentration
of 5.2 nM.

| | Target | Ct | Ct |
|---|---|---|---|
| MMLV RTase Variant | Name | Mean | SD |
| A646V | HPRT | 30.40 | 0.13 |
| | SFRS9 | 29.80 | 0.04 |
| G490A | HPRT | 31.23 | 0.13 |
| | SFRS9 | 30.75 | 0.06 |
| L333E | HPRT | 30.43 | 0.12 |
| | SFRS9 | 29.91 | 0.06 |
| K435H | HPRT | 31.08 | 0.25 |
| | SFRS9 | 30.35 | 0.40 |
| R159P | HPRT | 30.85 | 0.06 |
| | SFRS9 | 30.56 | 0.10 |
| R159S | HPRT | 31.16 | 0.04 |
| | SFRS9 | 30.78 | 0.07 |
| R629M | HPRT | 31.41 | 0.18 |
| | SFRS9 | 31.13 | 0.11 |
| T287D | HPRT | 30.49 | 0.17 |
| | SFRS9 | 30.04 | 0.09 |
| M63 Variant | HPRT | 31.54 | 0.32 |
| | SFRS9 | 31.19 | 0.12 |
| MMLV-II | HPRT | 40.00 | 0.00 |
| | SFRS9 | 38.31 | 0.56 |

TABLE 7
Two-Step cDNA synthesis by triple and quadruple
mutants of modified MMLV RTase. Data was generated
via qPCR assay for HPRT OR SFRS9, as indicated by
the “Target Name” column. Data is reported as Ct
values. Reaction temperatures were 65° C. and the
indicated MMLV RTase variant was at a concentration
of 5.2 nM.

| | Target | Ct | Ct |
|---|---|---|---|
| RTase | Name | Mean | SD |
| A646V | HPRT | 30.29 | 0.11 |
| | SFRS9 | 29.65 | 0.06 |
| G490A | HPRT | 30.89 | 0.11 |
| | SFRS9 | 30.29 | 0.06 |
| L333E | HPRT | 30.43 | 0.02 |
| | SFRS9 | 29.85 | 0.13 |
| K435H | HPRT | 31.27 | 0.28 |
| | SFRS9 | 30.65 | 0.36 |
| R159P | HPRT | 30.79 | 0.11 |
| | SFRS9 | 30.19 | 0.09 |
| R159S | HPRT | 30.89 | 0.08 |
| | SFRS9 | 30.28 | 0.04 |
| R629M | HPRT | 31.49 | 0.10 |
| | SFRS9 | 31.01 | 0.10 |
| T287D | HPRT | 30.35 | 0.04 |
| | SFRS9 | 29.80 | 0.06 |
| M63 Variant | HPRT | 31.63 | 0.12 |
| | SFRS9 | 30.99 | 0.07 |
| MMLV-II | HPRT | 39.50 | 0.71 |
| | SFRS9 | 37.85 | 0.07 |

TABLE 8
Two-Step cDNA synthesis by triple and quadruple
mutants of modified MMLV RTase. Data was generated
via qPCR assay for HPRT OR SFRS9, as indicated by
the “Target Name” column. Data is reported as Ct
values. Reaction temperatures were 65° C. and the
indicated MMLV RTase variant was at a concentration
of 0.52 nM.

| | Target | Ct | Ct |
|---|---|---|---|
| RTase | Name | Mean | SD |
| A646V | HPRT | 34.42 | 0.34 |
| | SFRS9 | 34.72 | 0.21 |
| G490A | HPRT | 35.59 | 1.86 |
| | SFRS9 | 34.84 | 0.28 |
| L333E | HPRT | 39.23 | 1.08 |
| | SFRS9 | 37.42 | 0.80 |
| K435H | HPRT | 39.08 | 1.30 |
| | SFRS9 | 37.86 | 1.19 |
| R159P | HPRT | 35.87 | 0.71 |
| | SFRS9 | 34.78 | 0.24 |
| R159S | HPRT | 35.94 | 0.59 |
| | SFRS9 | 36.21 | 0.74 |
| R629M | HPRT | 38.50 | 1.06 |
| | SFRS9 | 37.87 | 1.01 |
| T287D | HPRT | 35.74 | 0.23 |
| | SFRS9 | 35.67 | 0.56 |
| M63 Variant | HPRT | 36.74 | 0.49 |
| | SFRS9 | 36.79 | 0.56 |
| MMLV-II | HPRT | 36.85 | 0.74 |
| | SFRS9 | 36.02 | 0.33 |

### Example 4—Evaluation of MMLV Reverse Transcriptase Combination Mutants

Various combination mutants of the single mutants examined in Example 3 were generated to examine their activity and thermostability. In particular, T287D/L333E/K435H, T287D/L333E/G490, T287D/L333E/R629M, T287D/L333E/A646V, T287D/L333E/R159P, T287D/L333E/K435H/G490A, T287D/L333E/K435H/R629M, T287D/L333E/K435H/A646V, R159P/T287D/L333E/K435H, T287D/L333E/G490A/R629M, T287D/L333E/G490A/A646V, R159P/T287D/L333E/G490A, T287D/L333E/R629M/A646V, R159P/T287D/L333E/R629M, and R159P/T287D/L333E/A646V variants were generated by site directed mutagenesis according to the method described subsequently with respect to Example 1. All mutations were made to the M63 variant base sequence. The amino acid sequences of the above-indicated mutants as well as MMLV-II and the M63 variant MMLV RTase are provided in Table 9, below.

TABLE 9

| MMLV RTase | | |
|---|---|---|
| Variant Name | SEQ ID | Amino Acid Sequence (N-C) |
| M205-M63 + | 278 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| K435H | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNIHAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M206-M63 + | 279 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| G490A | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEALQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M207-M63 + | 280 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| R629M | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKMLSIIHCPGHQKGHSAEARGNRMAD |
| | | QAARKAAITETPDTSTLLIENSSPYTSEHF |
| M208-M63 + | 281 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| A646V | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEVRGNRMAD |
| | | QAARKAAITETPDTSTLLIENSSPYTSEHF |
| M20-M63 + | 282 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| R159P | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M210-M63 + | 283 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| K435H/G490A | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNIHAPHAVEALVKQPPDRWLSNARMTHYQALLLD |
| | | TDRVQFGPVVALNPATLLPLPEEALQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M211-M63 + | 284 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| K435H/R629M | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNIHAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKMLSIIHCPGHQKGHSAEARGNRMAD |
| | | QAARKAAITETPDTSTLLIENSSPYTSEHF* |
| M212-M63 + | 285 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| K435H/A646V | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNIHAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEVRGNRMAD |
| | | QAARKAAITETPDTSTLLIENSSPYTSEHF |
| M213-M63 + | 286 | MGSSHHHHHHSSGLVPRGSHMTLNIEDEHRLHETSKEP |
| R159P/T287D/ | | DVSLGSTWLSDFPQAWAETGGMGLAVRQAPLIIPLKAT |
| L333E/K435H | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNIHAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M214-M63 + | 287 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| G490A/R629M | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEALQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKMLSIIHCPGHQKGHSAEARGNRMAD |
| | | QAARKAAITETPDTSTLLIENSSPYTSEHF |
| M215-M63 + | 288 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| G490A/A646V | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEALQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEVRGNRMAD |
| | | QAARKAAITETPDTSTLLIENSSPYTSEHF |
| M216-M63 + | 289 | MGSSHHHHHHSSGLVPRGSHMTLNIEDEHRLHETSKEP |
| R159P/T287D/ | | DVSLGSTWLSDFPQAWAETGGMGLAVRQAPLIIPLKAT |
| L333E/G490A | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEALQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M217-M63 + | 290 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| R629M/A646V | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKMLSIIHCPGHQKGHSAEARGNRMAD |
| | | QAARKAAITETPDTSTLLIENSSPYTSEHF |
| M218-M63 + | 291 | MGSSHHHHHHSSGLVPRGSHMTLNIEDEHRLHETSKEP |
| R159P/T287D/ | | DVSLGSTWLSDFPQAWAETGGMGLAVRQAPLIIPLKAT |
| L333E/R629M | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKMLSIIHCPGHQKGHSAEARGNRMAD |
| | | QAARKAAITETPDTSTLLIENSSPYTSEHF |
| M219-M63 + | 292 | MGSSHHHHHHSSGLVPRGSHMTLNIEDEHRLHETSKEP |
| R159P/T287D/ | | DVSLGSTWLSDFPQAWAETGGMGLAVRQAPLIIPLKAT |
| L333E/A646V | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEVRGNRMAD |
| | | QAARKAAITETPDTSTLLIENSSPYTSEHF |

The ability of each combination mutant to synthesize cDNA from purified total Universal RNA (Agilent) was compared to purified protein having the sequence of SSII (Invitrogen; SEQ ID NO: 276) and the M63 variant base construct. In particular, the two-step assay described in the discussion of Example 3 was used with target temperatures of 50° C., 55° C., 60° C., 65° C. and a final concentration of reverse transcriptase of 5.2 nM. The two-step reactions for the MMLV variants were analyzed and reported by Ct value. Table 10 reports Ct values for each variant (including the M63 and MMLV-II variants) at each target temperature.

The T287D/L333E/K435H, T287D/L333E/G490, T287D/L333E/R629M, T287D/L333E/A646V, T287D/L333E/K435H/R629M, T287D/L333E/K435H/A646V, R159P/T287D/L333E/K435H, T287D/L333E/G490A/R629M, T287D/L333E/G490A/A646V, R159P/T287D/L333E/G490A, T287D/L333E/R629M/A646V, R159P/T287D/L333E/R629M, and R159P/T287D/L333E/A646V variants showed increased activity relative to both the M63 variant MMLV RTase and MMLV-II at all temperatures. The T287D/L333E/R159P and T287D/L333E/K435H/G490A variants showed comparable activity to the M63 variant RTase and improved activity over MMLV-II at all temperatures.

TABLE 10
Two-Step cDNA synthesis by triple and quadruple
mutants of modified MMLV RTase. Data was
generated via qPCR assay for HPRT OR
SFRS9, as indicated by the “Target Name”
column. Data is reported as Ct values.

| | cDNA | Target | Ct | Ct |
|---|---|---|---|---|
| MMLV RTase Variant | Temp | Name | Mean | SD |
| T287D/L333E/K435H | 50° C. | HPRT | 30.486 | 0.115 |
| | 55° C. | HPRT | 30.804 | 0.132 |
| | 60° C. | HPRT | 31.452 | 0.031 |
| | 65° C. | HPRT | 31.269 | 0.094 |
| | 50° C. | SFRS9 | 28.658 | 0.044 |
| | 55° C. | SFRS9 | 29.102 | 0.047 |
| | 60° C. | SFRS9 | 29.602 | 0.060 |
| | 65° C. | SFRS9 | 29.357 | 0.032 |
| T287D/L333E/G490A | 50° C. | HPRT | 30.610 | 0.134 |
| | 55° C. | HPRT | 31.285 | 0.091 |
| | 60° C. | HPRT | 31.790 | 0.048 |
| | 65° C. | HPRT | 31.362 | 0.074 |
| | 50° C. | SFRS9 | 28.969 | 0.031 |
| | 55° C. | SFRS9 | 29.806 | 0.048 |
| | 60° C. | SFRS9 | 30.190 | 0.182 |
| | 65° C. | SFRS9 | 29.612 | 0.046 |
| T287D/L333E/R629M | 50° C. | HPRT | 30.267 | 0.063 |
| | 55° C. | HPRT | 30.920 | 0.121 |
| | 60° C. | HPRT | 31.646 | 0.096 |
| | 65° C. | HPRT | 30.882 | 0.016 |
| | 50° C. | SFRS9 | 28.417 | 0.049 |
| | 55° C. | SFRS9 | 29.158 | 0.038 |
| | 60° C. | SFRS9 | 29.873 | 0.050 |
| | 65° C. | SFRS9 | 29.170 | 0.019 |
| T287D/L333E/A646V | 50° C. | HPRT | 30.378 | 0.133 |
| | 55° C. | HPRT | 30.985 | 0.029 |
| | 60° C. | HPRT | 31.592 | 0.226 |
| | 65° C. | HPRT | 31.228 | 0.074 |
| | 50° C. | SFRS9 | 28.650 | 0.066 |
| | 55° C. | SFRS9 | 29.375 | 0.063 |
| | 60° C. | SFRS9 | 29.982 | 0.170 |
| | 65° C. | SFRS9 | 29.331 | 0.084 |
| T287D/L333E/R159P | 50° C. | HPRT | 32.162 | 0.046 |
| | 55° C. | HPRT | 32.870 | 0.309 |
| | 60° C. | HPRT | 34.293 | 0.085 |
| | 65° C. | HPRT | 33.770 | 0.172 |
| | 50° C. | SFRS9 | 30.881 | 0.090 |
| | 55° C. | SFRS9 | 31.861 | 0.036 |
| | 60° C. | SFRS9 | 33.414 | 0.338 |
| | 65° C. | SFRS9 | 32.300 | 0.091 |
| T287D/L333E/K435H/G490A | 50° C. | HPRT | 32.486 | 0.151 |
| | 55° C. | HPRT | 32.958 | 0.115 |
| | 60° C. | HPRT | 34.279 | 0.273 |
| | 65° C. | HPRT | 34.533 | 0.143 |
| | 50° C. | SFRS9 | 30.878 | 0.094 |
| | 55° C. | SFRS9 | 31.482 | 0.065 |
| | 60° C. | SFRS9 | 32.755 | 0.083 |
| | 65° C. | SFRS9 | 32.626 | 0.147 |
| T287D/L333E/K435H/R629M | 50° C. | HPRT | 30.583 | 0.111 |
| | 55° C. | HPRT | 30.974 | 0.017 |
| | 60° C. | HPRT | 31.655 | 0.217 |
| | 65° C. | HPRT | 31.643 | 0.052 |
| | 50° C. | SFRS9 | 28.684 | 0.011 |
| | 55° C. | SFRS9 | 29.268 | 0.017 |
| | 60° C. | SFRS9 | 30.099 | 0.200 |
| | 65° C. | SFRS9 | 29.790 | 0.029 |
| T287D/L333E/K435H/A646V | 50° C. | HPRT | 30.050 | 0.074 |
| | 55° C. | HPRT | 30.932 | 0.106 |
| | 60° C. | HPRT | 31.316 | 0.074 |
| | 65° C. | HPRT | 31.511 | 0.109 |
| | 50° C. | SFRS9 | 28.181 | 0.035 |
| | 55° C. | SFRS9 | 29.133 | 0.024 |
| | 60° C. | SFRS9 | 29.790 | 0.083 |
| | 65° C. | SFRS9 | 29.665 | 0.084 |
| R159P/T287D/L333E/K435H | 50° C. | HPRT | 29.934 | 0.087 |
| | 55° C. | HPRT | 30.652 | 0.091 |
| | 60° C. | HPRT | 31.017 | 0.122 |
| | 65° C. | HPRT | 30.694 | 0.083 |
| | 50° C. | SFRS9 | 28.191 | 0.044 |
| | 55° C. | SFRS9 | 28.996 | 0.108 |
| | 60° C. | SFRS9 | 29.437 | 0.070 |
| | 65° C. | SFRS9 | 28.981 | 0.070 |
| T287D/L333E/G490A/R629M | 50° C. | HPRT | 30.323 | 0.075 |
| | 55° C. | HPRT | 31.170 | 0.131 |
| | 60° C. | HPRT | 31.541 | 0.164 |
| | 65° C. | HPRT | 31.211 | 0.160 |
| | 50° C. | SFRS9 | 28.460 | 0.036 |
| | 55° C. | SFRS9 | 29.450 | 0.019 |
| | 60° C. | SFRS9 | 29.880 | 0.163 |
| | 65° C. | SFRS9 | 29.454 | 0.042 |
| T287D/L333E/G490A/A646V | 50° C. | HPRT | 30.219 | 0.085 |
| | 55° C. | HPRT | 31.038 | 0.050 |
| | 60° C. | HPRT | 31.557 | 0.039 |
| | 65° C. | HPRT | 31.200 | 0.053 |
| | 50° C. | SFRS9 | 28.434 | 0.004 |
| | 55° C. | SFRS9 | 29.572 | 0.058 |
| | 60° C. | SFRS9 | 29.961 | 0.160 |
| | 65° C. | SFRS9 | 29.483 | 0.031 |
| R159P/T287D/L333E/G490A | 50° C. | HPRT | 29.779 | 0.183 |
| | 55° C. | HPRT | 30.765 | 0.102 |
| | 60° C. | HPRT | 30.892 | 0.062 |
| | 65° C. | HPRT | 30.557 | 0.056 |
| | 50° C. | SFRS9 | 28.084 | 0.161 |
| | 55° C. | SFRS9 | 29.142 | 0.047 |
| | 60° C. | SFRS9 | 29.208 | 0.076 |
| | 65° C. | SFRS9 | 28.735 | 0.012 |
| T287D/L333E/R629M/A646V | 50° C. | HPRT | 30.028 | 0.110 |
| | 55° C. | HPRT | 30.923 | 0.072 |
| | 60° C. | HPRT | 31.068 | 0.101 |
| | 65° C. | HPRT | 30.771 | 0.019 |
| | 50° C. | SFRS9 | 28.338 | 0.068 |
| | 55° C. | SFRS9 | 29.337 | 0.052 |
| | 60° C. | SFRS9 | 29.473 | 0.151 |
| | 65° C. | SFRS9 | 29.072 | 0.033 |
| R159P/T287D/L333E/R629M | 50° C. | HPRT | 30.191 | 0.144 |
| | 55° C. | HPRT | 31.094 | 0.077 |
| | 60° C. | HPRT | 31.190 | 0.166 |
| | 65° C. | HPRT | 30.900 | 0.062 |
| | 50° C. | SFRS9 | 28.509 | 0.107 |
| | 55° C. | SFRS9 | 29.525 | 0.064 |
| | 60° C. | SFRS9 | 29.532 | 0.074 |
| | 65° C. | SFRS9 | 29.300 | 0.085 |
| R159P/T287D/L333E/A646V | 50° C. | HPRT | 30.299 | 0.093 |
| | 55° C. | HPRT | 30.907 | 0.028 |
| | 60° C. | HPRT | 31.528 | 0.164 |
| | 65° C. | HPRT | 31.046 | 0.043 |
| | 50° C. | SFRS9 | 28.581 | 0.060 |
| | 55° C. | SFRS9 | 29.407 | 0.010 |
| | 60° C. | SFRS9 | 29.719 | 0.098 |
| | 65° C. | SFRS9 | 29.458 | 0.132 |
| M63 Variant | 50° C. | HPRT | 31.422 | 0.091 |
| | 55° C. | HPRT | 31.712 | 0.070 |
| | 60° C. | HPRT | 32.411 | 0.047 |
| | 65° C. | HPRT | 31.925 | 0.108 |
| | 50° C. | SFRS9 | 29.208 | 0.005 |
| | 55° C. | SFRS9 | 30.293 | 0.028 |
| | 60° C. | SFRS9 | 30.760 | 0.160 |
| | 65° C. | SFRS9 | 30.074 | 0.039 |
| MMLV-II | 50° C. | HPRT | 39.272 | 0.516 |
| | 55° C. | HPRT | 39.535 | 0.657 |
| | 60° C. | HPRT | 40.000 | 0.000 |
| | 65° C. | HPRT | 40.000 | 0.000 |
| | 50° C. | SFRS9 | 36.402 | 0.495 |
| | 55° C. | SFRS9 | 37.392 | 0.225 |
| | 60° C. | SFRS9 | 36.189 | 0.523 |
| | 65° C. | SFRS9 | 36.265 | 0.668 |

### Example 5—Evaluation of Additional MMLV Reverse Transcriptase Single Mutants

Various additional single mutants of the M63 variant base construct were generated to examine their activity and thermostability. In particular, A502STOP, A536STOP, A619L, C157A, D615P, D615R, E531S, E607S, E7P, F156V, G138S, G424S, G504A, H503R, I617V, K550W, L136A, K435H, L491T, L528V, L528Y, L604G, V433T, P196A, P196H, P196L, P401L, Q374L, Q530V, R173E, R9A, R9L, S137F, T427C, T467N, T505S, V129Y, V370S, V380S, and Y376I variants were generated by site directed mutagenesis according to the method described subsequently with respect to Example 1. All mutations were made to the M63 variant base sequence. The amino acid sequences of the above-indicated mutants are provided in Table 11, below.

TABLE 11

| MMLV RTase | | |
|---|---|---|
| Variant Name | SEQ ID | Amino Acid Sequence (N-C) |
| M63 + | 294 | MGSSHHHHHHSSGLVPRGSHMTLNIEDEHRLHETSKEP |
| A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAE |
| M63 + | 295 | MGSSHHHHHHSSGLVPRGSHMTLNIEDEHRLHETSKEP |
| A536STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRK |
| M63 + A619L | 296 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LLLLKALFLPKRLSIIHCPGHQKGHSAEARGNRMADQA |
| | | ARKAAITETPDTSTLLIENSSPYTSEHF |
| M63 + C157A | 297 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + D615P | 298 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKPEI |
| | | LALLKALFLPKRLSIIHCPGHQKGHSAEARGNRMADQA |
| | | ARKAAITETPDTSTLLIENSSPYTSEHF |
| M63 + D615R | 299 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKREI |
| | | LALLKALFLPKRLSIIHCPGHQKGHSAEARGNRMADQA |
| | | ARKAAITETPDTSTLLIENSSPYTSEHF |
| M63 + E531S | 300 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQSGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + E607S | 301 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSSGKEIKNKDEI |
| | | LALLKALFLPKRLSIIHCPGHQKGHSAEARGNRMADQA |
| | | ARKAAITETPDTSTLLIENSSPYTSEHF |
| M63 + E7P | 302 | MTLNIEDPHRLHETSKEPDVSLGSTWLSDFPQAWAETG |
| | | GMGLAVRQAPLIIPLKATSTPVSIKQYPMSREARLGIKP |
| | | HIRRLYDQGILVPCQSPWNTPLRPVKKPGTNDYRPVQD |
| | | LREVNKRVEDIHPTVPNPYNLLSGLPPSHQWYTVLDLK |
| | | DAFFCLRLHPTSQPLFAFEWRDPEMGISGQLTWTRLPQ |
| | | GFKNSPTLFDEALHRDLADFRIQHPDLILLQYVDDLLLA |
| | | ATSELDCQQGTRALLQTLGNLGYRASAKKAQICQKQV |
| | | KYLGYLLKEGQRWITDARKETVMGQPTPKTPRELREF |
| | | LGKAGFCRLWIPGFAEMAAPLYPLTKTGTLFNWGPDQ |
| | | QKAYQEIKQALLTAPALGLPDLTKPFELFVDEKQGYAK |
| | | GVLTQKLGPWRRPVAYLSKKLDPVAAGWPPCLRMVA |
| | | AIAVLTKDAGKLTMGQPLNILAPHAVEALVKQPPDRW |
| | | LSNARMTHYQALLLDTDRVQFGPVVALNPATLLPLPEE |
| | | GLQHNCLDILAEAHGTRPDLTDQPLPDADHTWYTGGS |
| | | SLLQEGQRKAGAAVTTETEVIWAKALPAGTSAQRAQLI |
| | | ALTQALKMAEGKKLNVYTNSRYAFATAHWHGEIYRR |
| | | RGLLTSEGKEIKNKDEILALLKALFLPKRLSIIHCPGHQK |
| | | GHSAEARGNRMADQAARKAAITETPDTSTLLIENSSPY |
| | | TSEHF |
| M63 + F156V | 303 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFVCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + G138S | 304 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSSLPPSHQWYTVLDLKDAFFCLRLHPTSQPLFAFE |
| | | WRDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + G424S | 305 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDASKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + G504A | 306 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHAT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + H503R | 307 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEARGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + I617V | 308 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDE |
| | | VLALLKALFLPKRLSIIHCPGHQKGHSAEARGNRMADQ |
| | | AARKAAITETPDTSTLLIENSSPYTSEHF |
| M63 + K550W | 309 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAWALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + L136A | 310 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLASGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + K435H | 311 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + L491T | 312 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGTQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + L528V | 313 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSVLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + L528Y | 314 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + L604G | 315 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLGTSEGKEIKNKDEI |
| | | LALLKALFLPKRLSIIHCPGHQKGHSAEARGNRMADQA |
| | | ARKAAITETPDTSTLLIENSSPYTSEHF |
| M63 + V433T | 316 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + P196A | 317 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSATLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + P196H | 318 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSHTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + P196L | 319 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSLTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + P401L | 320 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDLVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + Q374L | 321 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKLGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + Q530V | 322 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLVEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + R173E | 323 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + R9A | 324 | MGSSHHHHHHSSGLVPRGSHMTLNIEDEHALHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + R9L | 325 | MGSSHHHHHHSSGLVPRGSHMTLNIEDEHLLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + S137F | 326 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLFGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + T427C | 327 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLCMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + T467N | 328 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + T505S | 329 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGS |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + V129Y | 330 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTYPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + V370S | 331 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFSDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + V380S | 332 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGSLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + Y376I | 333 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGIAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |

The ability of each combination mutant to synthesize cDNA from purified total Universal RNA (Agilent) was compared to purified protein having the sequence of SSII (Invitrogen; SEQ ID NO: 276) and the M63 variant base construct. In particular, the two-step assay described in the discussion of Example 3 was used with target temperatures of 50° C., 55° C., 60° C., 65° C. and a final concentration of reverse transcriptase of 5.2 nM. The two-step reactions for the MMLV variants were analyzed and reported by Ct value. Table 12 reports Ct values for each variant (including the M63 and MMLV-II variants) at 50° C. and 55° C., and Table 13 Ct values for each variant (including the M63 and MMLV-II variants) at 60° C. and 65° C.

As is shown in the data of Table 12 and Table 13, all mutants showed significantly improved activity as compared to the MMLV-II variant at all examined temperatures. Further, all mutants showed comparable or improved activity as compared to the M63 variant MMLV RTase. In particular, the R9A (SEQ ID NO: 324), C157A (SEQ ID NO: 297), R173E (SEQ ID NO: 323), V433T (SEQ ID NO: 316), T467N (SEQ ID NO: 328), A502STOP (SEQ ID NO: 294), and L528Y (SEQ ID NO: 314) variants showed significantly increased activity at multiple temperatures (as determined by at least one of the HPRT and SFRS9 assays) as compared to both M63 and MMLV-II variants. Notably, the M63 variant also includes a mutation to valine 433 (V433N). Advantageously, the V433T mutant shows improved activity relative to the M63 variant at 50° C., 55° C., 60° C., and 65° C. P

TABLE 12
Two-Step cDNA synthesis by single mutants of modified
MMLV RTase. Data was generated via qPCR assay for
HPRT OR SFRS9, as indicated by the “Target Name”
column. Data is reported as Ct values.

| | cDNA | Target | Ct | Ct |
|---|---|---|---|---|
| MMLV RTase Variant | Temp | Name | Mean | SD |
| A436S | 50° C. | HPRT | 31.156 | 0.178 |
| | 55° C. | HPRT | 32.338 | 0.170 |
| | 50° C. | SFRS9 | 30.158 | 0.084 |
| | 55° C. | SFRS9 | 31.784 | 0.086 |
| A502STOP | 50° C. | HPRT | 29.704 | 0.108 |
| | 55° C. | HPRT | 31.389 | 0.095 |
| | 50° C. | SFRS9 | 29.302 | 0.079 |
| | 55° C. | SFRS9 | 30.827 | 0.093 |
| A536STOP | 50° C. | HPRT | 30.485 | 0.190 |
| | 55° C. | HPRT | 31.401 | 0.092 |
| | 50° C. | SFRS9 | 29.966 | 0.057 |
| | 55° C. | SFRS9 | 31.340 | 0.150 |
| A619L | 50° C. | HPRT | 30.666 | 0.226 |
| | 55° C. | HPRT | 31.714 | 0.175 |
| | 50° C. | SFRS9 | 29.629 | 0.032 |
| | 55° C. | SFRS9 | 31.508 | 0.088 |
| C157A | 50° C. | HPRT | 29.816 | 0.096 |
| | 55° C. | HPRT | 31.025 | 0.120 |
| | 50° C. | SFRS9 | 29.103 | 0.115 |
| | 55° C. | SFRS9 | 30.763 | 0.136 |
| D615P | 50° C. | HPRT | 30.876 | 0.073 |
| | 55° C. | HPRT | 31.984 | 0.051 |
| | 50° C. | SFRS9 | 29.864 | 0.006 |
| | 55° C. | SFRS9 | 31.829 | 0.105 |
| D615R | 50° C. | HPRT | 31.402 | 0.105 |
| | 55° C. | HPRT | 32.369 | 0.074 |
| | 50° C. | SFRS9 | 30.204 | 0.084 |
| | 55° C. | SFRS9 | 31.917 | 0.181 |
| E531S | 50° C. | HPRT | 30.812 | 0.137 |
| | 55° C. | HPRT | 31.420 | 0.143 |
| | 50° C. | SFRS9 | 29.816 | 0.104 |
| | 55° C. | SFRS9 | 30.951 | 0.181 |
| E607S | 50° C. | HPRT | 31.354 | 0.212 |
| | 55° C. | HPRT | 31.934 | 0.062 |
| | 50° C. | SFRS9 | 30.019 | 0.048 |
| | 55° C. | SFRS9 | 31.431 | 0.148 |
| E7P | 50° C. | HPRT | 30.943 | 0.145 |
| | 55° C. | HPRT | 31.538 | 0.073 |
| | 50° C. | SFRS9 | 29.689 | 0.017 |
| | 55° C. | SFRS9 | 31.449 | 0.022 |
| F156V | 50° C. | HPRT | 32.270 | 0.125 |
| | 55° C. | HPRT | 33.306 | 0.219 |
| | 50° C. | SFRS9 | 31.279 | 0.289 |
| | 55° C. | SFRS9 | 33.544 | 0.149 |
| G138S | 50° C. | HPRT | 30.507 | 0.124 |
| | 55° C. | HPRT | 31.388 | 0.061 |
| | 50° C. | SFRS9 | 29.535 | 0.138 |
| | 55° C. | SFRS9 | 31.167 | 0.174 |
| G424S | 50° C. | HPRT | 30.435 | 0.084 |
| | 55° C. | HPRT | 31.640 | 0.042 |
| | 50° C. | SFRS9 | 29.593 | 0.116 |
| | 55° C. | SFRS9 | 31.151 | 0.131 |
| G504A | 50° C. | HPRT | 31.016 | 0.142 |
| | 55° C. | HPRT | 31.174 | 0.221 |
| | 50° C. | SFRS9 | 29.998 | 0.081 |
| | 55° C. | SFRS9 | 30.712 | 0.106 |
| H503R | 50° C. | HPRT | 31.112 | 0.069 |
| | 55° C. | HPRT | 32.074 | 0.257 |
| | 50° C. | SFRS9 | 30.146 | 0.197 |
| | 55° C. | SFRS9 | 31.517 | 0.155 |
| I617V | 50° C. | HPRT | 30.686 | 0.209 |
| | 55° C. | HPRT | 31.541 | 0.083 |
| | 50° C. | SFRS9 | 29.447 | 0.029 |
| | 55° C. | SFRS9 | 31.257 | 0.244 |
| K550W | 50° C. | HPRT | 30.441 | 0.098 |
| | 55° C. | HPRT | 31.475 | 0.220 |
| | 50° C. | SFRS9 | 29.624 | 0.021 |
| | 55° C. | SFRS9 | 31.078 | 0.078 |
| L136A | 50° C. | HPRT | 32.445 | 0.206 |
| | 55° C. | HPRT | 33.547 | 0.120 |
| | 50° C. | SFRS9 | 31.468 | 0.071 |
| | 55° C. | SFRS9 | 33.390 | 0.018 |
| K435H | 50° C. | HPRT | 30.535 | 0.248 |
| | 55° C. | HPRT | 31.196 | 0.141 |
| | 50° C. | SFRS9 | 29.538 | 0.008 |
| | 55° C. | SFRS9 | 30.830 | 0.055 |
| L491T | 50° C. | HPRT | 31.233 | 0.095 |
| | 55° C. | HPRT | 31.978 | 0.082 |
| | 50° C. | SFRS9 | 30.344 | 0.054 |
| | 55° C. | SFRS9 | 32.040 | 0.179 |
| L528V | 50° C. | HPRT | 31.442 | 0.107 |
| | 55° C. | HPRT | 31.910 | 0.191 |
| | 50° C. | SFRS9 | 30.550 | 0.093 |
| | 55° C. | SFRS9 | 31.477 | 0.048 |
| L528Y | 50° C. | HPRT | 30.632 | 0.087 |
| | 55° C. | HPRT | 31.827 | 0.106 |
| | 50° C. | SFRS9 | 29.401 | 0.052 |
| | 55° C. | SFRS9 | 31.366 | 0.111 |
| L604G | 50° C. | HPRT | 30.604 | 0.007 |
| | 55° C. | HPRT | 31.806 | 0.299 |
| | 50° C. | SFRS9 | 29.617 | 0.084 |
| | 55° C. | SFRS9 | 31.271 | 0.135 |
| MMLV-II | 50° C. | HPRT | 40.000 | 0.000 |
| | 55° C. | HPRT | 40.000 | 0.000 |
| | 50° C. | SFRS9 | 36.936 | 0.850 |
| | 55° C. | SFRS9 | 39.315 | 0.889 |
| M63 Variant | 50° C. | HPRT | 30.734 | 0.059 |
| | 55° C. | HPRT | 31.402 | 0.040 |
| | 50° C. | SFRS9 | 29.675 | 0.038 |
| | 55° C. | SFRS9 | 31.237 | 0.044 |
| V433T | 50° C. | HPRT | 30.246 | 0.171 |
| | 55° C. | HPRT | 30.746 | 0.042 |
| | 50° C. | SFRS9 | 29.188 | 0.100 |
| | 55° C. | SFRS9 | 30.328 | 0.134 |
| P196A | 50° C. | HPRT | 31.709 | 0.156 |
| | 55° C. | HPRT | 32.805 | 0.158 |
| | 50° C. | SFRS9 | 31.200 | 0.117 |
| | 55° C. | SFRS9 | 33.013 | 0.243 |
| P196H | 50° C. | HPRT | 31.775 | 0.315 |
| | 55° C. | HPRT | 33.340 | 0.153 |
| | 50° C. | SFRS9 | 31.576 | 0.055 |
| | 55° C. | SFRS9 | 33.208 | 0.198 |
| P196L | 50° C. | HPRT | 30.780 | 0.097 |
| | 55° C. | HPRT | 32.401 | 0.227 |
| | 50° C. | SFRS9 | 30.413 | 0.047 |
| | 55° C. | SFRS9 | 32.569 | 0.081 |
| P401L | 50° C. | HPRT | 32.747 | 0.152 |
| | 55° C. | HPRT | 33.794 | 0.501 |
| | 50° C. | SFRS9 | 31.935 | 0.050 |
| | 55° C. | SFRS9 | 33.788 | 0.320 |
| Q374L | 50° C. | HPRT | 31.317 | 0.064 |
| | 55° C. | HPRT | 32.628 | 0.172 |
| | 50° C. | SFRS9 | 30.416 | 0.116 |
| | 55° C. | SFRS9 | 32.123 | 0.232 |
| Q530V | 50° C. | HPRT | 33.361 | 0.270 |
| | 55° C. | HPRT | 35.522 | 0.721 |
| | 50° C. | SFRS9 | 32.713 | 0.107 |
| | 55° C. | SFRS9 | 34.506 | 0.246 |
| R173E | 50° C. | HPRT | 29.998 | 0.158 |
| | 55° C. | HPRT | 31.010 | 0.166 |
| | 50° C. | SFRS9 | 29.448 | 0.044 |
| | 55° C. | SFRS9 | 30.999 | 0.131 |
| R9A | 50° C. | HPRT | 30.298 | 0.113 |
| | 55° C. | HPRT | 31.237 | 0.112 |
| | 50° C. | SFRS9 | 29.285 | 0.035 |
| | 55° C. | SFRS9 | 30.754 | 0.110 |
| R9L | 50° C. | HPRT | 31.199 | 0.161 |
| | 55° C. | HPRT | 31.820 | 0.204 |
| | 50° C. | SFRS9 | 30.188 | 0.077 |
| | 55° C. | SFRS9 | 31.907 | 0.176 |
| S137F | 50° C. | HPRT | 31.641 | 0.023 |
| | 55° C. | HPRT | 32.731 | 0.219 |
| | 50° C. | SFRS9 | 30.676 | 0.107 |
| | 55° C. | SFRS9 | 32.405 | 0.024 |
| T427C | 50° C. | HPRT | 31.752 | 0.175 |
| | 55° C. | HPRT | 32.274 | 0.233 |
| | 50° C. | SFRS9 | 30.586 | 0.041 |
| | 55° C. | SFRS9 | 32.395 | 0.149 |
| T467N | 50° C. | HPRT | 30.225 | 0.021 |
| | 55° C. | HPRT | 31.120 | 0.197 |
| | 50° C. | SFRS9 | 29.362 | 0.089 |
| | 55° C. | SFRS9 | 30.955 | 0.198 |
| T505S | 50° C. | HPRT | 31.255 | 0.091 |
| | 55° C. | HPRT | 32.112 | 0.178 |
| | 50° C. | SFRS9 | 30.371 | 0.080 |
| | 55° C. | SFRS9 | 31.948 | 0.020 |
| V129Y | 50° C. | HPRT | 31.615 | 0.035 |
| | 55° C. | HPRT | 32.819 | 0.278 |
| | 50° C. | SFRS9 | 30.470 | 0.265 |
| | 55° C. | SFRS9 | 32.502 | 0.112 |
| V370S | 50° C. | HPRT | 35.407 | 0.394 |
| | 55° C. | HPRT | 38.654 | 1.904 |
| | 50° C. | SFRS9 | 34.462 | 0.114 |
| | 55° C. | SFRS9 | 37.876 | 1.931 |
| V380S | 50° C. | HPRT | 32.767 | 0.134 |
| | 55° C. | HPRT | 33.886 | 0.136 |
| | 50° C. | SFRS9 | 32.219 | 0.086 |
| | 55° C. | SFRS9 | 33.767 | 0.184 |
| Y376I | 50° C. | HPRT | 31.325 | 0.061 |
| | 55° C. | HPRT | 32.540 | 0.331 |
| | 50° C. | SFRS9 | 30.340 | 0.051 |
| | 55° C. | SFRS9 | 32.130 | 0.109 |

TABLE 13
Two-Step cDNA synthesis by single mutants of
modified MMLV RTase. Data was generated via qPCR
assay for HPRT OR SFRS9, as indicated by the
“Target Name” column. Data is reported as Ct values.

| | cDNA | Target | Ct | Ct |
|---|---|---|---|---|
| MMLV RTase Variant | Temp | Name | Mean | SD |
| A436S | 60° C. | HPRT | 31.731 | 0.278 |
| | 65° C. | HPRT | 32.519 | 0.305 |
| | 60° C. | SFRS9 | 32.960 | 0.132 |
| | 65° C. | SFRS9 | 33.279 | 0.191 |
| A502STOP | 60° C. | HPRT | 33.361 | 0.551 |
| | 65° C. | HPRT | 33.984 | 0.176 |
| | 60° C. | SFRS9 | 32.748 | 0.374 |
| | 65° C. | SFRS9 | 33.040 | 0.122 |
| A536STOP | 60° C. | HPRT | 31.984 | 0.019 |
| | 65° C. | HPRT | 34.310 | 0.660 |
| | 60° C. | SFRS9 | 32.501 | 0.313 |
| | 65° C. | SFRS9 | 33.668 | 0.376 |
| A619L | 60° C. | HPRT | 32.067 | 0.000 |
| | 65° C. | HPRT | 33.061 | 0.230 |
| | 60° C. | SFRS9 | 33.125 | 0.889 |
| | 65° C. | SFRS9 | 33.301 | 0.279 |
| C157A | 60° C. | HPRT | 30.593 | 0.060 |
| | 65° C. | HPRT | 30.683 | 0.225 |
| | 60° C. | SFRS9 | 31.248 | 0.147 |
| | 65° C. | SFRS9 | 31.767 | 0.216 |
| D615P | 60° C. | HPRT | 32.614 | 0.498 |
| | 65° C. | HPRT | 33.940 | 0.395 |
| | 60° C. | SFRS9 | 32.788 | 0.082 |
| | 65° C. | SFRS9 | 33.710 | 0.239 |
| D615R | 60° C. | HPRT | 32.236 | 0.062 |
| | 65° C. | HPRT | 34.047 | 0.683 |
| | 60° C. | SFRS9 | 32.992 | 0.138 |
| | 65° C. | SFRS9 | 33.775 | 0.650 |
| E531S | 60° C. | HPRT | 31.463 | 0.365 |
| | 65° C. | HPRT | 32.386 | 0.215 |
| | 60° C. | SFRS9 | 32.193 | 0.217 |
| | 65° C. | SFRS9 | 32.756 | 0.222 |
| E607S | 60° C. | HPRT | 31.393 | 0.105 |
| | 65° C. | HPRT | 32.349 | 0.719 |
| | 60° C. | SFRS9 | 32.384 | 0.120 |
| | 65° C. | SFRS9 | 32.993 | 0.344 |
| E7P | 60° C. | HPRT | 31.617 | 0.163 |
| | 65° C. | HPRT | 32.247 | 0.266 |
| | 60° C. | SFRS9 | 32.172 | 0.203 |
| | 65° C. | SFRS9 | 33.015 | 0.283 |
| F156V | 60° C. | HPRT | 33.830 | 0.038 |
| | 65° C. | HPRT | 36.150 | 2.737 |
| | 60° C. | SFRS9 | 35.000 | 0.485 |
| | 65° C. | SFRS9 | 37.584 | 1.810 |
| G138S | 60° C. | HPRT | 31.423 | 0.196 |
| | 65° C. | HPRT | 32.009 | 0.453 |
| | 60° C. | SFRS9 | 32.225 | 0.005 |
| | 65° C. | SFRS9 | 32.871 | 0.177 |
| G424S | 60° C. | HPRT | 31.021 | 0.451 |
| | 65° C. | HPRT | 33.381 | 0.823 |
| | 60° C. | SFRS9 | 32.342 | 0.158 |
| | 65° C. | SFRS9 | 33.948 | 0.617 |
| G504A | 60° C. | HPRT | 32.078 | 0.632 |
| | 65° C. | HPRT | 33.188 | 0.577 |
| | 60° C. | SFRS9 | 32.544 | 0.185 |
| | 65° C. | SFRS9 | 34.508 | 0.555 |
| H503R | 60° C. | HPRT | 31.671 | 0.431 |
| | 65° C. | HPRT | 32.834 | 0.541 |
| | 60° C. | SFRS9 | 32.847 | 0.109 |
| | 65° C. | SFRS9 | 33.373 | 0.178 |
| I617V | 60° C. | HPRT | 30.928 | 0.383 |
| | 65° C. | HPRT | 32.212 | 0.666 |
| | 60° C. | SFRS9 | 31.790 | 0.048 |
| | 65° C. | SFRS9 | 33.312 | 0.269 |
| K550W | 60° C. | HPRT | 30.983 | 0.303 |
| | 65° C. | HPRT | 32.524 | 0.270 |
| | 60° C. | SFRS9 | 32.140 | 0.161 |
| | 65° C. | SFRS9 | 32.873 | 0.064 |
| L136A | 60° C. | HPRT | 33.733 | 0.289 |
| | 65° C. | HPRT | 34.707 | 0.663 |
| | 60° C. | SFRS9 | 34.960 | 0.555 |
| | 65° C. | SFRS9 | 35.916 | 1.108 |
| K435H | 60° C. | HPRT | 31.601 | 0.541 |
| | 65° C. | HPRT | 32.513 | 0.196 |
| | 60° C. | SFRS9 | 32.134 | 0.176 |
| | 65° C. | SFRS9 | 33.464 | 0.261 |
| L491T | 60° C. | HPRT | 31.859 | 0.455 |
| | 65° C. | HPRT | 32.907 | 0.777 |
| | 60° C. | SFRS9 | 33.131 | 0.214 |
| | 65° C. | SFRS9 | 33.761 | 0.466 |
| L528V | 60° C. | HPRT | 31.544 | 0.022 |
| | 65° C. | HPRT | 32.679 | 0.120 |
| | 60° C. | SFRS9 | 32.599 | 0.192 |
| | 65° C. | SFRS9 | 33.699 | 0.271 |
| L528Y | 60° C. | HPRT | 30.756 | 0.088 |
| | 65° C. | HPRT | 31.846 | 0.651 |
| | 60° C. | SFRS9 | 31.695 | 0.042 |
| | 65° C. | SFRS9 | 32.307 | 0.173 |
| L604G | 60° C. | HPRT | 32.995 | 0.493 |
| | 65° C. | HPRT | 35.138 | 0.956 |
| | 60° C. | SFRS9 | 32.745 | 0.136 |
| | 65° C. | SFRS9 | 33.955 | 0.141 |
| MMLV-II | 60° C. | HPRT | 40.000 | 0.000 |
| | 65° C. | HPRT | 40.000 | 0.000 |
| | 60° C. | SFRS9 | 37.581 | 0.870 |
| | 65° C. | SFRS9 | 36.984 | 0.422 |
| M63 Variant | 60° C. | HPRT | 30.858 | 0.189 |
| | 65° C. | HPRT | 32.182 | 0.289 |
| | 60° C. | SFRS9 | 31.793 | 0.075 |
| | 65° C. | SFRS9 | 33.278 | 0.193 |
| V433T | 60° C. | HPRT | 30.326 | 0.067 |
| | 65° C. | HPRT | 31.855 | 0.353 |
| | 60° C. | SFRS9 | 31.561 | 0.114 |
| | 65° C. | SFRS9 | 32.620 | 0.164 |
| P196A | 60° C. | HPRT | 34.207 | 0.193 |
| | 65° C. | HPRT | 36.194 | 0.209 |
| | 60° C. | SFRS9 | 34.614 | 0.223 |
| | 65° C. | SFRS9 | 35.902 | 1.173 |
| P196H | 60° C. | HPRT | 35.810 | 0.459 |
| | 65° C. | HPRT | 36.612 | 2.407 |
| | 60° C. | SFRS9 | 35.249 | 0.421 |
| | 65° C. | SFRS9 | 35.989 | 0.498 |
| P196L | 60° C. | HPRT | 32.766 | 0.426 |
| | 65° C. | HPRT | 34.145 | 1.717 |
| | 60° C. | SFRS9 | 33.498 | 0.256 |
| | 65° C. | SFRS9 | 34.127 | 0.548 |
| P401L | 60° C. | HPRT | 32.972 | 0.486 |
| | 65° C. | HPRT | 33.634 | 0.575 |
| | 60° C. | SFRS9 | 34.478 | 0.504 |
| | 65° C. | SFRS9 | 34.537 | 0.191 |
| Q374L | 60° C. | HPRT | 32.656 | 0.615 |
| | 65° C. | HPRT | 33.415 | 0.546 |
| | 60° C. | SFRS9 | 33.467 | 0.290 |
| | 65° C. | SFRS9 | 34.399 | 0.343 |
| Q530V | 60° C. | HPRT | 33.815 | 0.425 |
| | 65° C. | HPRT | 34.956 | 0.624 |
| | 60° C. | SFRS9 | 35.200 | 0.555 |
| | 65° C. | SFRS9 | 36.259 | 0.421 |
| R173E | 60° C. | HPRT | 31.211 | 0.290 |
| | 65° C. | HPRT | 31.513 | 0.189 |
| | 60° C. | SFRS9 | 32.178 | 0.064 |
| | 65° C. | SFRS9 | 32.481 | 0.068 |
| R9A | 60° C. | HPRT | 30.775 | 0.052 |
| | 65° C. | HPRT | 31.782 | 0.401 |
| | 60° C. | SFRS9 | 31.537 | 0.101 |
| | 65° C. | SFRS9 | 32.478 | 0.023 |
| R9L | 60° C. | HPRT | 31.648 | 0.306 |
| | 65° C. | HPRT | 34.292 | 1.344 |
| | 60° C. | SFRS9 | 32.768 | 0.311 |
| | 65° C. | SFRS9 | 33.965 | 0.260 |
| S137F | 60° C. | HPRT | 33.357 | 0.163 |
| | 65° C. | HPRT | 35.658 | 0.675 |
| | 60° C. | SFRS9 | 33.571 | 0.069 |
| | 65° C. | SFRS9 | 35.028 | 0.370 |
| T427C | 60° C. | HPRT | 32.056 | 0.313 |
| | 65° C. | HPRT | 34.923 | 0.979 |
| | 60° C. | SFRS9 | 33.533 | 0.078 |
| | 65° C. | SFRS9 | 34.649 | 0.284 |
| T467N | 60° C. | HPRT | 31.989 | 0.356 |
| | 65° C. | HPRT | 33.114 | 0.301 |
| | 60° C. | SFRS9 | 32.624 | 0.124 |
| | 65° C. | SFRS9 | 33.135 | 0.207 |
| T505S | 60° C. | HPRT | 32.766 | 0.217 |
| | 65° C. | HPRT | 33.316 | 0.345 |
| | 60° C. | SFRS9 | 32.922 | 0.195 |
| | 65° C. | SFRS9 | 33.424 | 0.267 |
| V129Y | 60° C. | HPRT | 32.998 | 0.559 |
| | 65° C. | HPRT | 34.176 | 0.163 |
| | 60° C. | SFRS9 | 33.911 | 0.140 |
| | 65° C. | SFRS9 | 34.535 | 0.185 |
| V370S | 60° C. | HPRT | 35.659 | 0.779 |
| | 65° C. | HPRT | 37.014 | 2.166 |
| | 60° C. | SFRS9 | 38.050 | 0.842 |
| | 65° C. | SFRS9 | 36.458 | 0.411 |
| V380S | 60° C. | HPRT | 33.963 | 1.162 |
| | 65° C. | HPRT | 35.138 | 0.360 |
| | 60° C. | SFRS9 | 35.229 | 0.453 |
| | 65° C. | SFRS9 | 34.984 | 0.171 |
| Y376I | 60° C. | HPRT | 33.342 | 0.904 |
| | 65° C. | HPRT | 34.003 | 0.701 |
| | 60° C. | SFRS9 | 32.997 | 0.164 |
| | 65° C. | SFRS9 | 33.912 | 0.129 |

### Example 6—Evaluation of Additional MMLV Reverse Transcriptase Single Mutants

A further set of single mutants of the M63 variant base construct were generated to examine their activity and thermostability. In particular, A477Y, A587T, G524N, P474S, T523L, and W593L variants were generated by site directed mutagenesis according to the method described subsequently with respect to Example 1. All mutations were made to the M63 variant base sequence. The amino acid sequences of the generated mutants are provided in Table 14, below.

TABLE 14

| MMLV RTase | | |
|---|---|---|
| Variant Name | SEQ ID | Amino Acid Sequence (N-C) |
| M63 + A477Y | 294 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVYLNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + A587T | 295 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYTFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + G524N | 296 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTNGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + P474S | 297 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGSVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + T523L | 298 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYLGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + W593L | 299 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHLHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |

The ability of each combination mutant to synthesize cDNA from purified total Universal RNA (Agilent) was compared to purified protein having the sequence of SSII (Invitrogen; SEQ ID NO: 276) and the M63 variant base construct. In particular, the two-step assay described in the discussion of Example 3 was used with target temperatures of 50° C., 55° C., 60° C., 65° C. and a final concentration of reverse transcriptase of 5.2 nM. The two-step reactions for the MMLV variants were analyzed and reported by Ct value. Table 12 reports Ct values for each variant (including the M63 and MMLV-II variants) at 50° C. and 55° C., and Table 13 Ct values for each variant (including the M63 and MMLV-II variants) at 60° C. and 65° C.

As is shown in the data of Table 15, all examined mutants showed significantly improved activity as compared to the MMLV-II variant at all examined temperatures. Further, all mutants showed comparable activity as compared to the M63 variant MMLV RTase.

TABLE 15
Two-Step cDNA synthesis by triple and quadruple mutants
of modified MMLV RTase. Data was generated via qPCR
assay for HPRT OR SFRS9, as indicated by the “Target
Name” column. Data is reported as Ct values.

| | cDNA | Target | Ct | Ct |
|---|---|---|---|---|
| MMLV RTase Variant | Temp | Name | Mean | SD |
| A477Y | 50° C. | HPRT | 32.487 | 0.231 |
| | 55° C. | HPRT | 33.616 | 0.104 |
| | 60° C. | HPRT | 34.897 | 0.277 |
| | 65° C. | HPRT | 35.872 | 0.441 |
| | 50° C. | SFRS9 | 31.958 | 0.149 |
| | 55° C. | SFRS9 | 33.505 | 0.170 |
| | 60° C. | SFRS9 | 34.283 | 0.229 |
| | 65° C. | SFRS9 | 35.025 | 0.226 |
| A587T | 50° C. | HPRT | 31.129 | 0.238 |
| | 55° C. | HPRT | 31.810 | 0.055 |
| | 60° C. | HPRT | 33.289 | 0.184 |
| | 65° C. | HPRT | 34.014 | 0.336 |
| | 50° C. | SFRS9 | 30.249 | 0.080 |
| | 55° C. | SFRS9 | 31.659 | 0.093 |
| | 60° C. | SFRS9 | 32.636 | 0.022 |
| | 65° C. | SFRS9 | 33.224 | 0.126 |
| G524N | 50° C. | HPRT | 31.275 | 0.166 |
| | 55° C. | HPRT | 32.201 | 0.104 |
| | 60° C. | HPRT | 32.845 | 0.064 |
| | 65° C. | HPRT | 33.619 | 0.164 |
| | 50° C. | SFRS9 | 30.344 | 0.035 |
| | 55° C. | SFRS9 | 31.711 | 0.121 |
| | 60° C. | SFRS9 | 32.664 | 0.171 |
| | 65° C. | SFRS9 | 33.201 | 0.037 |
| M63 Variant | 50° C. | HPRT | 31.079 | 0.021 |
| | 55° C. | HPRT | 31.521 | 0.136 |
| | 60° C. | HPRT | 32.599 | 0.190 |
| | 65° C. | HPRT | 33.020 | 0.257 |
| | 50° C. | SFRS9 | 30.022 | 0.108 |
| | 55° C. | SFRS9 | 31.255 | 0.192 |
| | 60° C. | SFRS9 | 32.043 | 0.156 |
| | 65° C. | SFRS9 | 32.701 | 0.061 |
| P474S | 50° C. | HPRT | 31.997 | 0.026 |
| | 55° C. | HPRT | 32.704 | 0.091 |
| | 60° C. | HPRT | 33.897 | 0.272 |
| | 65° C. | HPRT | 34.074 | 0.086 |
| | 50° C. | SFRS9 | 31.491 | 0.045 |
| | 55° C. | SFRS9 | 32.824 | 0.081 |
| | 60° C. | SFRS9 | 33.147 | 0.082 |
| | 65° C. | SFRS9 | 33.863 | 0.176 |
| T523L | 50° C. | HPRT | 31.019 | 0.108 |
| | 55° C. | HPRT | 31.633 | 0.115 |
| | 60° C. | HPRT | 32.845 | 0.195 |
| | 65° C. | HPRT | 33.481 | 0.235 |
| | 50° C. | SFRS9 | 30.035 | 0.050 |
| | 55° C. | SFRS9 | 31.219 | 0.133 |
| | 60° C. | SFRS9 | 32.222 | 0.142 |
| | 65° C. | SFRS9 | 32.955 | 0.028 |
| W593L | 50° C. | HPRT | 30.896 | 0.121 |
| | 55° C. | HPRT | 31.990 | 0.086 |
| | 60° C. | HPRT | 34.203 | 0.333 |
| | 65° C. | HPRT | 34.147 | 0.272 |
| | 50° C. | SFRS9 | 30.234 | 0.052 |
| | 55° C. | SFRS9 | 32.140 | 0.085 |
| | 60° C. | SFRS9 | 33.323 | 0.198 |
| | 65° C. | SFRS9 | 34.002 | 0.228 |
| MMLV-II | 50° C. | HPRT | 40.000 | 0.000 |
| | 55° C. | HPRT | 40.000 | 0.000 |
| | 60° C. | HPRT | 40.000 | 0.000 |
| | 65° C. | HPRT | 39.952 | 0.068 |
| | 50° C. | SFRS9 | 37.108 | 0.273 |
| | 55° C. | SFRS9 | 38.016 | 0.183 |
| | 60° C. | SFRS9 | 39.708 | 0.299 |
| | 65° C. | SFRS9 | 38.310 | 0.253 |

### Example 6—Evaluation of Additional MMLV Reverse Transcriptase Single Mutants and Further Combination Mutants

A further set of single mutants of the M63 variant base construct were generated as were a set of combination mutants of advantageous mutants identified Example 3 and Example 5. In particular, A477Y, A587T, G524N, P474S, T523L, and W593L variants were generated by site directed mutagenesis according to the method described subsequently with respect to Example 1. All mutations were made to the M63 variant base sequence. The amino acid sequences of the generated mutants are provided in Table 16, below.

TABLE 16

| MMLV RTase | | |
|---|---|---|
| Variant Name | SEQ ID | Amino Acid Sequence (N-C) |
| M63 + A500Q | 340 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILQEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + A619R | 341 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LRLLKALFLPKRLSIIHCPGHQKGHSAEARGNRMADQA |
| | | ARKAAITETPDTSTLLIENSSPYTSEHF |
| M63 + R629T | 342 | MGSSHHHHHHSSGLVPRGSHMTLNIEDEHRLHETSKEP |
| | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKTLSIIHCPGHQKGHSAEARGNRMADQA |
| | | ARKAAITETPDTSTLLIENSSPYTSEHF |
| M63 + | 343 | MGSSHHHHHHSSGLVPRGSHMTLNIEDEHALHETSKEP |
| R9A/C157A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 344 | MGSSHHHHHHSSGLVPRGSHMTLNIEDEHRLHETSKEP |
| C157A/R159P | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 345 | MGSSHHHHHHSSGLVPRGSHMTLNIEDEHRLHETSKEP |
| C157A/R159S | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 346 | MGSSHHHHHHSSGLVPRGSHMTLNIEDEHRLHETSKEP |
| C157A/R173E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 347 | MGSSHHHHHHSSGLVPRGSHMTLNIEDEHRLHETSKEP |
| C157A/T287D | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 348 | MGSSHHHHHHSSGLVPRGSHMTLNIEDEHRLHETSKEP |
| C157A/L333E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 349 | MGSSHHHHHHSSGLVPRGSHMTLNIEDEHRLHETSKEP |
| C157A/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 350 | MGSSHHHHHHSSGLVPRGSHMTLNIEDEHRLHETSKEP |
| C157A/K435H | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 351 | MGSSHHHHHHSSGLVPRGSHMTLNIEDEHRLHETSKEP |
| C157A/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 352 | MGSSHHHHHHSSGLVPRGSHMTLNIEDEHRLHETSKEP |
| C157A/G490A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEALQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 353 | MGSSHHHHHHSSGLVPRGSHMTLNIEDEHRLHETSKEP |
| C157A/G490A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEALQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 354 | MGSSHHHHHHSSGLVPRGSHMTLNIEDEHRLHETSKEP |
| C157A/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAE |
| M63 + | 355 | MGSSHHHHHHSSGLVPRGSHMTLNIEDEHRLHETSKEP |
| C157A/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 356 | MGSSHHHHHHSSGLVPRGSHMTLNIEDEHRLHETSKEP |
| C157A/R629M | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKMLSIIHCPGHQKGHSAEARGNRMADQ |
| | | AARKAAITETPDTSTLLIENSSPYTSEHF |
| M63 + | 357 | MGSSHHHHHHSSGLVPRGSHMTLNIEDEHRLHETSKEP |
| C157A/A646V | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEVRGNRMADQA |
| | | ARKAAITETPDTSTLLIENSSPYTSEHF |
| M63 + | 358 | MGSSHHHHHHSSGLVPRGSHMTLNIEDEHALHETSKEP |
| R9A/R159P | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 359 | MGSSHHHHHHSSGLVPRGSHMTLNIEDEHALHETSKEP |
| R9A/R159S | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 360 | MGSSHHHHHHSSGLVPRGSHMTLNIEDEHALHETSKEP |
| R9A/R173E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 361 | MGSSHHHHHHSSGLVPRGSHMTLNIEDEHALHETSKEP |
| R9A/T287D | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 362 | MGSSHHHHHHSSGLVPRGSHMTLNIEDEHALHETSKEP |
| R9A/L333E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 363 | MGSSHHHHHHSSGLVPRGSHMTLNIEDEHALHETSKEP |
| R9A/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 364 | MGSSHHHHHHSSGLVPRGSHMTLNIEDEHALHETSKEP |
| R9A/K435H | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 365 | MGSSHHHHHHSSGLVPRGSHMTLNIEDEHALHETSKEP |
| R9A/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 366 | MGSSHHHHHHSSGLVPRGSHMTLNIEDEHALHETSKEP |
| R9A/G490A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEALQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 367 | MGSSHHHHHHSSGLVPRGSHMTLNIEDEHALHETSKEP |
| R9A/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAE |
| M63 + | 368 | MGSSHHHHHHSSGLVPRGSHMTLNIEDEHALHETSKEP |
| R9A/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 369 | MGSSHHHHHHSSGLVPRGSHMTLNIEDEHALHETSKEP |
| R9A/R629M | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKMLSIIHCPGHQKGHSAEARGNRMADQ |
| | | AARKAAITETPDTSTLLIENSSPYTSEHF |
| M63 + | 370 | MGSSHHHHHHSSGLVPRGSHMTLNIEDEHALHETSKEP |
| R9A/A646V | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEVRGNRMADQA |
| | | ARKAAITETPDTSTLLIENSSPYTSEHF |
| M63 + | 371 | MGSSHHHHHHSSGLVPRGSHMTLNIEDEHRLHETSKEP |
| R159P/R173E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 372 | MGSSHHHHHHSSGLVPRGSHMTLNIEDEHRLHETSKEP |
| R159P/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 373 | MGSSHHHHHHSSGLVPRGSHMTLNIEDEHRLHETSKEP |
| R159P/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 374 | MGSSHHHHHHSSGLVPRGSHMTLNIEDEHRLHETSKEP |
| R159P/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAE |
| M63 + | 375 | MGSSHHHHHHSSGLVPRGSHMTLNIEDEHRLHETSKEP |
| R159P/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLPLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 376 | MGSSHHHHHHSSGLVPRGSHMTLNIEDEHRLHETSKEP |
| R159S/R173E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 377 | MGSSHHHHHHSSGLVPRGSHMTLNIEDEHRLHETSKEP |
| R159S/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 378 | MGSSHHHHHHSSGLVPRGSHMTLNIEDEHRLHETSKEP |
| R159S/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 379 | MGSSHHHHHHSSGLVPRGSHMTLNIEDEHRLHETSKEP |
| R159S/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAE |
| M63 + | 380 | MGSSHHHHHHSSGLVPRGSHMTLNIEDEHRLHETSKEP |
| R159S/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLSLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 381 | MGSSHHHHHHSSGLVPRGSHMTLNIEDEHRLHETSKEP |
| R173E/T287D | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKEDVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 382 | MGSSHHHHHHSSGLVPRGSHMTLNIEDEHRLHETSKEP |
| R173E/L333E | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTEFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 383 | MGSSHHHHHHSSGLVPRGSHMTLNIEDEHRLHETSKEP |
| R173E/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LTILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 384 | MGSSHHHHHHSSGLVPRGSHMTLNIEDEHRLHETSKEP |
| R173E/K435H | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNIHAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 385 | MGSSHHHHHHSSGLVPRGSHMTLNIEDEHRLHETSKEP |
| R173E/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDND |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 386 | MGSSHHHHHHSSGLVPRGSHMTLNIEDEHRLHETSKEP |
| R173E/G490A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEALQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 387 | MGSSHHHHHHSSGLVPRGSHMTLNIEDEHRLHETSKEP |
| R173E/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAE |
| M63 + | 388 | MGSSHHHHHHSSGLVPRGSHMTLNIEDEHRLHETSKEP |
| R173E/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSYLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEARGNRMADQA |
| | | ARKAAITETPDTSTLLIENSSPYTSEHF |
| M63 + | 389 | MGSSHHHHHHSSGLVPRGSHMTLNIEDEHRLHETSKEP |
| R173E/R629M | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKMLSIIHCPGHQKGHSAEARGNRMADQA |
| | | ARKAAITETPDTSTLLIENSSPYTSEHF |
| M63 + | 390 | MGSSHHHHHHSSGLVPRGSHMTLNIEDEHRLHETSKEP |
| R173E/A646V | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKETVMGQPTPKTPRELREFLGKAGFCRLWIPGFAEMA |
| | | APLYPLTKTGTLFNWGPDQQKAYQEIKQALLTAPALG |
| | | LPDLTKPFELFVDEKQGYAKGVLTQKLGPWRRPVAYL |
| | | SKKLDPVAAGWPPCLRMVAAIAVLTKDAGKLTMGQP |
| | | LNILAPHAVEALVKQPPDRWLSNARMTHYQALLLDTD |
| | | RVQFGPVVALNPATLLPLPEEGLQHNCLDILAEAHGTR |
| | | PDLTDQPLPDADHTWYTGGSSLLQEGQRKAGAAVTTE |
| | | TEVIWAKALPAGTSAQRAQLIALTQALKMAEGKKLNV |
| | | YTNSRYAFATAHWHGEIYRRRGLLTSEGKEIKNKDEIL |
| | | ALLKALFLPKRLSIIHCPGHQKGHSAEVRGNRMADQA |
| | | ARKAAITETPDTSTLLIENSSPYTSEHF |
| M63 + | 391 | MGSSHHHHHHSSGLVPRGSHMTLNIEDEHRLHETSKEP |
| T287D/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLTILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 392 | MGSSHHHHHHSSGLVPRGSHMTLNIEDEHRLHETSKEP |
| T287D/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | NDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 393 | MGSSHHHHHHSSGLVPRGSHMTLNIEDEHRLHETSKEP |
| T287D/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAE |
| M63 + | 394 | MGSSHHHHHHSSGLVPRGSHMTLNIEDEHRLHETSKEP |
| T287D/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTLFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSYLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 395 | MGSSHHHHHHSSGLVPRGSHMTLNIEDEHRLHETSKEP |
| L333E/V433T | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 396 | MGSSHHHHHHSSGLVPRGSHMTLNIEDEHRLHETSKEP |
| L333E/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 397 | MGSSHHHHHHSSGLVPRGSHMTLNIEDEHRLHETSKEP |
| L333E/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAE |
| M63 + | 398 | MGSSHHHHHHSSGLVPRGSHMTLNIEDEHRLHETSKEP |
| L333E/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 399 | MGSSHHHHHHSSGLVPRGSHMTLNIEDEHRLHETSKEP |
| V433T/K435H | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTIHAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 400 | MGSSHHHHHHSSGLVPRGSHMTLNIEDEHRLHETSKEP |
| V433T/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 401 | MGSSHHHHHHSSGLVPRGSHMTLNIEDEHRLHETSKEP |
| V433T/G490A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEALQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 402 | MGSSHHHHHHSSGLVPRGSHMTLNIEDEHRLHETSKEP |
| V433T/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAE |
| M63 + | 403 | MGSSHHHHHHSSGLVPRGSHMTLNIEDEHRLHETSKEP |
| V433T/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 404 | MGSSHHHHHHSSGLVPRGSHMTLNIEDEHRLHETSKEP |
| V433T/R629M | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKMLSIIHCPGHQKGHSAEARGNRMADQ |
| | | AARKAAITETPDTSTLLIENSSPYTSEHF |
| M63 + | 405 | MGSSHHHHHHSSGLVPRGSHMTLNIEDEHRLHETSKEP |
| V433T/A646V | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLTILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEVRGNRMADQA |
| | | ARKAAITETPDTSTLLIENSSPYTSEHF |
| M63 + | 406 | MGSSHHHHHHSSGLVPRGSHMTLNIEDEHRLHETSKEP |
| K435H/T467N | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 407 | MGSSHHHHHHSSGLVPRGSHMTLNIEDEHRLHETSKEP |
| K435H/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDT |
| | | DRVQFGPVVALNPATLLPLPEEGLQHNCLDILAE |
| M63 + | 408 | MGSSHHHHHHSSGLVPRGSHMTLNIEDEHRLHETSKEP |
| K435H/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNIHAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 409 | MGSSHHHHHHSSGLVPRGSHMTLNIEDEHRLHETSKEP |
| T467N/G490A | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEALQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 410 | MGSSHHHHHHSSGLVPRGSHMTLNIEDEHRLHETSKEP |
| T467N/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAE |
| M63 + | 411 | MGSSHHHHHHSSGLVPRGSHMTLNIEDEHRLHETSKEP |
| T467N/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 412 | MGSSHHHHHHSSGLVPRGSHMTLNIEDEHRLHETSKEP |
| T467N/R629M | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKMLSIIHCPGHQKGHSAEARGNRMADQ |
| | | AARKAAITETPDTSTLLIENSSPYTSEHF |
| M63 + | 413 | MGSSHHHHHHSSGLVPRGSHMTLNIEDEHRLHETSKEP |
| T467N/A646V | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDN |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEVRGNRMADQA |
| | | ARKAAITETPDTSTLLIENSSPYTSEHF |
| M63 + | 414 | MGSSHHHHHHSSGLVPRGSHMTLNIEDEHRLHETSKEP |
| G490A/A502STOP | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEALQHNCLDILAE |
| M63 + | 415 | MGSSHHHHHHSSGLVPRGSHMTLNIEDEHRLHETSKEP |
| G490A/L528Y | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEALQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 416 | MGSSHHHHHHSSGLVPRGSHMTLNIEDEHRLHETSKEP |
| L528Y/R629M | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKMLSIIHCPGHQKGHSAEARGNRMADQ |
| | | AARKAAITETPDTSTLLIENSSPYTSEHF |
| M63 + | 417 | MGSSHHHHHHSSGLVPRGSHMTLNIEDEHRLHETSKEP |
| L528Y/A646V | | DVSLGSTWLSDFPQAWAETGGMGLAVRQAPLIIPLKAT |
| | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKETVMGQPTPKTPRELREFLGKAGFCRLWIPGFAEM |
| | | AAPLYPLTKTGTLFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSYLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEVRGNRMADQA |
| | | ARKAAITETPDTSTLLIENSSPYTSEHF |
| M63 + | 418 | MGSSHHHHHHSSGLVPRGSHMTLNIEDEHALHETSKEP |
| R9A/T287D/ | | DVSLGSTWLSDFPQAWAETGGMGLAVRQAPLIIPLKAT |
| L333E | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 419 | MGSSHHHHHHSSGLVPRGSHMTLNIEDEHRLHETSKEP |
| C157A/T287D/ | | DVSLGSTWLSDFPQAWAETGGMGLAVRQAPLIIPLKAT |
| L333E | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFALRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 420 | MGSSHHHHHHSSGLVPRGSHMTLNIEDEHRLHETSKEP |
| R173E/T287D/ | | DVSLGSTWLSDFPQAWAETGGMGLAVRQAPLIIPLKAT |
| L333E | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWEDPEMGISGQLTWTRLPQGFKNSPTLFDEALHRDLA |
| | | DFRIQHPDLILLQYVDDLLLAATSELDCQQGTRALLQT |
| | | LGNLGYRASAKKAQICQKQVKYLGYLLKEGQRWITDA |
| | | RKEDVMGQPTPKTPRELREFLGKAGFCRLWIPGFAEM |
| | | AAPLYPLTKTGTEFNWGPDQQKAYQEIKQALLTAPAL |
| | | GLPDLTKPFELFVDEKQGYAKGVLTQKLGPWRRPVAY |
| | | LSKKLDPVAAGWPPCLRMVAAIAVLTKDAGKLTMGQ |
| | | PLNILAPHAVEALVKQPPDRWLSNARMTHYQALLLDT |
| | | DRVQFGPVVALNPATLLPLPEEGLQHNCLDILAEAHGT |
| | | RPDLTDQPLPDADHTWYTGGSSLLQEGQRKAGAAVTT |
| | | ETEVIWAKALPAGTSAQRAQLIALTQALKMAEGKKLN |
| | | VYTNSRYAFATAHWHGEIYRRRGLLTSEGKEIKNKDEI |
| | | LALLKALFLPKRLSIIHCPGHQKGHSAEARGNRMADQA |
| | | ARKAAITETPDTSTLLIENSSPYTSEHF |
| M63 + | 421 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| V433T | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLTILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 422 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| T467N | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | NDRVQFGPVVALNPATLLPLPEEGLQHNCLDILAEAHG |
| | | TRPDLTDQPLPDADHTWYTGGSSLLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |
| M63 + | 423 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| A502STOP | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAE |
| M63 + | 424 | MGSSHHHHHHSSGLVPRGSHMTLNIEDEHRLHETSKEP |
| T287D/L333E/ | | DVSLGSTWLSDFPQAWAETGGMGLAVRQAPLIIPLKAT |
| L528Y | | STPVSIKQYPMSREARLGIKPHIRRLYDQGILVPCQSPW |
| | | NTPLRPVKKPGTNDYRPVQDLREVNKRVEDIHPTVPNP |
| | | YNLLSGLPPSHQWYTVLDLKDAFFCLRLHPTSQPLFAF |
| | | EWRDPEMGISGQLTWTRLPQGFKNSPTLFDEALHRDL |
| | | ADFRIQHPDLILLQYVDDLLLAATSELDCQQGTRALLQ |
| | | TLGNLGYRASAKKAQICQKQVKYLGYLLKEGQRWITD |
| | | ARKEDVMGQPTPKTPRELREFLGKAGFCRLWIPGFAE |
| | | MAAPLYPLTKTGTEFNWGPDQQKAYQEIKQALLTAPA |
| | | LGLPDLTKPFELFVDEKQGYAKGVLTQKLGPWRRPVA |
| | | YLSKKLDPVAAGWPPCLRMVAAIAVLTKDAGKLTMG |
| | | QPLNILAPHAVEALVKQPPDRWLSNARMTHYQALLLD |
| | | TDRVQFGPVVALNPATLLPLPEEGLQHNCLDILAEAHG |
| | | TRPDLTDQPLPDADHTWYTGGSSYLQEGQRKAGAAVT |
| | | TETEVIWAKALPAGTSAQRAQLIALTQALKMAEGKKL |
| | | NVYTNSRYAFATAHWHGEIYRRRGLLTSEGKEIKNKD |
| | | EILALLKALFLPKRLSIIHCPGHQKGHSAEARGNRMAD |
| | | QAARKAAITETPDTSTLLIENSSPYTSEHF |

The ability of each mutant to synthesize cDNA from purified total Universal RNA (Agilent) was compared to purified protein having the sequence of SSII (Invitrogen; SEQ ID NO: 276) and the M63 variant base construct. In particular, the two-step assay described in the discussion of Example 3 was used with target temperatures of 50° C., 55° C., 60° C., 65° C. and a final concentration of reverse transcriptase of 5.2 nM. The two-step reactions for the MMLV variants were analyzed and reported by Ct value. Table 17, Table 18, Table 19, and Table 20 report Ct values for each variant (including the M63 and MMLV-II variants) at 50° C., 55° C., 60° C., and 65° C., respectively.

As is shown by the data of Tables 17-20, all mutants showed significantly improved activity as compared to the MMLV-II variant at all examined temperatures. Further, all mutants showed comparable or improved activity as compared to the M63 variant MMLV RTase at all examined temperatures. The stacked variants having the highest activity and greatest thermostability were T287D/L333E (SEQ ID NO: 418), C157A/T287D (SEQ ID NO: 419), R9A/T287D (SEQ ID NO: 361), R9A/V433T (SEQ ID NO: 363), T287D/V433T (SEQ ID NO: 391), T287D/A502STOP (SEQ ID NO: 393), V433T/A502STOP (SEQ ID NO: 402), T287D/L333E/V433T (SEQ ID NO: 421), T287D/L333E/A502STOP (SEQ ID NO: 423), and T287D/L333E/L528Y (SEQ ID NO: 424). variants showed significantly increased activity at multiple temperatures (i.e., and improved thermostability) as compared to both M63 and MMLV-II variants.

TABLE 17
Two-Step cDNA synthesis by single, double, and triple
mutants of modified MMLV RTase. Data was generated
via qPCR assay for HPRT OR SFRS9, as indicated
by the “Target Name” column.

| | cDNA | Target | Ct | Ct |
|---|---|---|---|---|
| MMLV RTase Variant | Temp | Name | Mean | SD |
| A500Q | 50° C. | HPRT | 31.759 | 0.058 |
| | 50° C. | SFRS9 | 31.221 | 0.025 |
| A619R | 50° C. | HPRT | 31.967 | 0.109 |
| | 50° C. | SFRS9 | 31.460 | 0.070 |
| R629T | 50° C. | HPRT | 31.654 | 0.145 |
| | 50° C. | HPRT | 31.911 | 0.064 |
| R9A/C157A | 50° C. | SFRS9 | 31.185 | 0.074 |
| | 50° C. | SFRS9 | 31.412 | 0.046 |
| C157A/R159P | 50° C. | HPRT | 31.923 | 0.037 |
| | 50° C. | SFRS9 | 32.143 | 0.159 |
| C157A/R159S | 50° C. | HPRT | 32.767 | 0.147 |
| | 50° C. | SFRS9 | 32.653 | 0.128 |
| C157A/R173E | 50° C. | HPRT | 30.766 | 0.066 |
| | 50° C. | SFRS9 | 30.524 | 0.074 |
| C157A/T287D | 50° C. | HPRT | 30.012 | 0.011 |
| | 50° C. | SFRS9 | 29.690 | 0.054 |
| C157A/L333E | 50° C. | HPRT | 30.754 | 0.049 |
| | 50° C. | SFRS9 | 30.153 | 0.106 |
| C157A/V433T | 50° C. | HPRT | 30.535 | 0.112 |
| | 50° C. | SFRS9 | 30.048 | 0.077 |
| C157A/K435H | 50° C. | HPRT | 31.129 | 0.103 |
| | 50° C. | SFRS9 | 30.582 | 0.025 |
| C157A/T467N | 50° C. | HPRT | 31.348 | 0.100 |
| | 50° C. | SFRS9 | 30.818 | 0.030 |
| C157A/G490A | 50° C. | HPRT | 30.742 | 0.116 |
| | 50° C. | SFRS9 | 30.324 | 0.153 |
| C157A/A502STOP | 50° C. | HPRT | 30.192 | 0.104 |
| | 50° C. | SFRS9 | 29.954 | 0.013 |
| C157A/L528Y | 50° C. | HPRT | 31.288 | 0.133 |
| | 50° C. | SFRS9 | 30.812 | 0.037 |
| C157A/R629M | 50° C. | HPRT | 30.868 | 0.053 |
| | 50° C. | SFRS9 | 30.287 | 0.026 |
| C157A/A646V | 50° C. | HPRT | 30.876 | 0.019 |
| | 50° C. | SFRS9 | 30.364 | 0.044 |
| R9A/R159P | 50° C. | HPRT | 30.624 | 0.044 |
| | 50° C. | SFRS9 | 30.202 | 0.117 |
| R9A/R173E | 50° C. | HPRT | 30.676 | 0.120 |
| | 50° C. | SFRS9 | 30.299 | 0.044 |
| R9A/T287D | 50° C. | HPRT | 30.149 | 0.066 |
| | 50° C. | SFRS9 | 29.718 | 0.036 |
| R9A/L333E | 50° C. | HPRT | 30.609 | 0.095 |
| | 50° C. | SFRS9 | 29.971 | 0.032 |
| R9A/V433T | 50° C. | HPRT | 30.281 | 0.028 |
| | 50° C. | SFRS9 | 29.528 | 0.042 |
| R9A/K435H | 50° C. | HPRT | 30.699 | 0.155 |
| | 50° C. | SFRS9 | 30.019 | 0.035 |
| R9A/T467N | 50° C. | HPRT | 31.267 | 0.045 |
| | 50° C. | SFRS9 | 30.582 | 0.106 |
| R9A/G490A | 50° C. | HPRT | 30.868 | 0.040 |
| | 50° C. | SFRS9 | 30.241 | 0.076 |
| R9A/A502STOP | 50° C. | HPRT | 30.270 | 0.063 |
| | 50° C. | SFRS9 | 29.897 | 0.071 |
| R9A/L528Y | 50° C. | HPRT | 30.980 | 0.034 |
| | 50° C. | SFRS9 | 30.467 | 0.064 |
| R9A/R629M | 50° C. | HPRT | 30.913 | 0.137 |
| | 50° C. | SFRS9 | 30.305 | 0.068 |
| R9A/A646V | 50° C. | HPRT | 31.141 | 0.068 |
| | 50° C. | SFRS9 | 30.651 | 0.081 |
| R159P/R173E | 50° C. | HPRT | 30.877 | 0.083 |
| | 50° C. | SFRS9 | 30.438 | 0.015 |
| R159P/V433T | 50° C. | HPRT | 30.044 | 0.050 |
| | 50° C. | SFRS9 | 29.642 | 0.023 |
| R159P/T467N | 50° C. | HPRT | 30.908 | 0.103 |
| | 50° C. | SFRS9 | 30.330 | 0.028 |
| R159P/A502STOP | 50° C. | HPRT | 30.007 | 0.015 |
| | 50° C. | SFRS9 | 29.697 | 0.048 |
| R159P/L528Y | 50° C. | HPRT | 30.772 | 0.035 |
| | 50° C. | SFRS9 | 30.365 | 0.122 |
| R159S/R173E | 50° C. | HPRT | 31.630 | 0.166 |
| | 50° C. | SFRS9 | 31.497 | 0.021 |
| R159S/V433T | 50° C. | HPRT | 31.072 | 0.133 |
| | 50° C. | SFRS9 | 30.504 | 0.209 |
| R159S/T467N | 50° C. | HPRT | 32.079 | 0.090 |
| | 50° C. | SFRS9 | 31.483 | 0.065 |
| R159S/A502STOP | 50° C. | HPRT | 30.300 | 0.058 |
| | 50° C. | SFRS9 | 30.163 | 0.084 |
| R159S/L528Y | 50° C. | HPRT | 31.307 | 0.087 |
| | 50° C. | SFRS9 | 30.816 | 0.100 |
| R173E/T287D | 50° C. | HPRT | 30.457 | 0.146 |
| | 50° C. | SFRS9 | 30.075 | 0.116 |
| R173E/L333E | 50° C. | HPRT | 30.522 | 0.102 |
| | 50° C. | SFRS9 | 29.999 | 0.082 |
| R173E/V433T | 50° C. | HPRT | 30.864 | 0.079 |
| | 50° C. | SFRS9 | 30.295 | 0.078 |
| R173E/K435H | 50° C. | HPRT | 30.635 | 0.136 |
| | 50° C. | SFRS9 | 30.064 | 0.072 |
| R173E/T467N | 50° C. | HPRT | 30.956 | 0.041 |
| | 50° C. | SFRS9 | 30.556 | 0.114 |
| R173E/G490A | 50° C. | HPRT | 31.304 | 0.171 |
| | 50° C. | SFRS9 | 30.909 | 0.145 |
| R173E/A502STOP | 50° C. | HPRT | 30.862 | 0.100 |
| | 50° C. | SFRS9 | 30.776 | 0.040 |
| R173E/L528Y | 50° C. | HPRT | 30.973 | 0.132 |
| | 50° C. | SFRS9 | 30.587 | 0.094 |
| R173E/R629M | 50° C. | HPRT | 31.457 | 0.093 |
| | 50° C. | SFRS9 | 31.041 | 0.015 |
| R173E/A646V | 50° C. | HPRT | 30.891 | 0.073 |
| | 50° C. | SFRS9 | 30.495 | 0.069 |
| T287D/V433T | 50° C. | HPRT | 29.810 | 0.096 |
| | 50° C. | SFRS9 | 29.368 | 0.038 |
| T287D/T467N | 50° C. | HPRT | 30.562 | 0.077 |
| | 50° C. | SFRS9 | 29.978 | 0.035 |
| T287D/A502STOP | 50° C. | HPRT | 29.865 | 0.078 |
| | 50° C. | SFRS9 | 29.486 | 0.048 |
| T287D/L528Y | 50° C. | HPRT | 30.519 | 0.070 |
| | 50° C. | SFRS9 | 30.038 | 0.053 |
| L333E/V433T | 50° C. | HPRT | 30.394 | 0.105 |
| | 50° C. | SFRS9 | 29.813 | 0.032 |
| L333E/T467N | 50° C. | HPRT | 31.009 | 0.070 |
| | 50° C. | SFRS9 | 30.306 | 0.054 |
| L333E/A502STOP | 50° C. | HPRT | 30.470 | 0.104 |
| | 50° C. | SFRS9 | 30.200 | 0.073 |
| L333E/L528Y | 50° C. | HPRT | 30.474 | 0.071 |
| | 50° C. | SFRS9 | 29.733 | 0.060 |
| V433T/K435H | 50° C. | HPRT | 30.708 | 0.110 |
| | 50° C. | SFRS9 | 29.992 | 0.101 |
| V433T/T467N | 50° C. | HPRT | 31.233 | 0.172 |
| | 50° C. | SFRS9 | 30.507 | 0.046 |
| V433T/G490A | 50° C. | HPRT | 31.121 | 0.205 |
| | 50° C. | SFRS9 | 30.475 | 0.089 |
| V433T/A502STOP | 50° C. | HPRT | 29.896 | 0.076 |
| | 50° C. | SFRS9 | 29.219 | 0.070 |
| V433T/L528Y | 50° C. | HPRT | 31.168 | 0.123 |
| | 50° C. | SFRS9 | 30.449 | 0.021 |
| V433T/R629M | 50° C. | HPRT | 31.449 | 0.072 |
| | 50° C. | SFRS9 | 30.742 | 0.081 |
| V433T/A646V | 50° C. | HPRT | 30.895 | 0.136 |
| | 50° C. | SFRS9 | 30.371 | 0.053 |
| K435H/T467N | 50° C. | HPRT | 31.331 | 0.078 |
| | 50° C. | SFRS9 | 30.732 | 0.135 |
| K435H/A502STOP | 50° C. | HPRT | 30.225 | 0.119 |
| | 50° C. | SFRS9 | 29.640 | 0.012 |
| K435H/L528Y | 50° C. | HPRT | 31.207 | 0.042 |
| | 50° C. | SFRS9 | 30.374 | 0.032 |
| T467N/G490A | 50° C. | HPRT | 31.588 | 0.082 |
| | 50° C. | SFRS9 | 30.796 | 0.073 |
| T467N/A502STOP | 50° C. | HPRT | 30.358 | 0.071 |
| | 50° C. | SFRS9 | 29.987 | 0.039 |
| T467N/L528Y | 50° C. | HPRT | 30.837 | 0.038 |
| | 50° C. | SFRS9 | 30.106 | 0.082 |
| T467N/R629M | 50° C. | HPRT | 31.630 | 0.109 |
| | 50° C. | SFRS9 | 31.006 | 0.101 |
| T467N/A646V | 50° C. | HPRT | 31.742 | 0.080 |
| | 50° C. | SFRS9 | 31.002 | 0.028 |
| G490A/A502STOP | 50° C. | HPRT | 30.668 | 0.036 |
| | 50° C. | SFRS9 | 30.271 | 0.102 |
| G490A/L528Y | 50° C. | HPRT | 31.364 | 0.095 |
| | 50° C. | SFRS9 | 30.679 | 0.089 |
| L528Y/R629M | 50° C. | HPRT | 30.956 | 0.080 |
| | 50° C. | SFRS9 | 30.344 | 0.082 |
| L528Y/A646V | 50° C. | HPRT | 31.682 | 0.132 |
| | 50° C. | SFRS9 | 30.988 | 0.117 |
| R9A/T287D/L333E | 50° C. | HPRT | 30.110 | 0.044 |
| | 50° C. | SFRS9 | 29.788 | 0.073 |
| C157A/T287D/L333E | 50° C. | HPRT | 29.961 | 0.036 |
| | 50° C. | SFRS9 | 29.652 | 0.039 |
| R173E/T287D/L333E | 50° C. | HPRT | 30.330 | 0.053 |
| | 50° C. | SFRS9 | 29.999 | 0.075 |
| T287D/L333E/V433T | 50° C. | HPRT | 30.063 | 0.064 |
| | 50° C. | SFRS9 | 29.589 | 0.068 |
| T287D/L333E/T467N | 50° C. | HPRT | 29.970 | 0.044 |
| | 50° C. | SFRS9 | 29.529 | 0.014 |
| T287D/L333E/A502STOP | 50° C. | HPRT | 29.853 | 0.068 |
| | 50° C. | SFRS9 | 29.506 | 0.023 |
| T287D/L333E/L528Y | 50° C. | HPRT | 29.972 | 0.093 |
| | 50° C. | SFRS9 | 29.440 | 0.047 |
| Mutant from App | 50° C. | HPRT | 31.326 | 0.015 |
| #20230031558 | 50° C. | SFRS9 | 30.818 | 0.150 |
| MMLV-II | 50° C. | HPRT | 35.881 | 0.338 |
| | 50° C. | SFRS9 | 36.255 | 0.082 |

TABLE 18
Two-Step cDNA synthesis by single, double, and triple
mutants of modified MMLV RTase. Data was generated
via qPCR assay for HPRT OR SFRS9, as indicated by the
“Target Name” column. Data is reported as Ct values.

| | cDNA | Target | Ct | Ct |
|---|---|---|---|---|
| MMLV RTase Variant | Temp | Name | Mean | SD |
| A500Q | 55° C. | HPRT | 32.914 | 0.222 |
| | 55° C. | SFRS9 | 32.836 | 0.052 |
| A619R | 55° C. | HPRT | 32.915 | 0.044 |
| | 55° C. | SFRS9 | 32.602 | 0.075 |
| R629T | 55° C. | HPRT | 32.725 | 0.196 |
| | 55° C. | HPRT | 32.976 | 0.049 |
| R9A/C157A | 55° C. | SFRS9 | 32.369 | 0.040 |
| | 55° C. | SFRS9 | 32.399 | 0.178 |
| C157A/R159P | 55° C. | HPRT | 33.166 | 0.066 |
| | 55° C. | SFRS9 | 32.572 | 0.087 |
| C157A/R159S | 55° C. | HPRT | 34.530 | 0.232 |
| | 55° C. | SFRS9 | 33.903 | 0.094 |
| C157A/R173E | 55° C. | HPRT | 31.814 | 0.179 |
| | 55° C. | SFRS9 | 31.341 | 0.084 |
| C157A/T287D | 55° C. | HPRT | 30.791 | 0.083 |
| | 55° C. | SFRS9 | 30.258 | 0.088 |
| C157A/L333E | 55° C. | HPRT | 31.249 | 0.080 |
| | 55° C. | SFRS9 | 30.942 | 0.033 |
| C157A/V433T | 55° C. | HPRT | 31.344 | 0.060 |
| | 55° C. | SFRS9 | 30.726 | 0.026 |
| C157A/K435H | 55° C. | HPRT | 32.251 | 0.094 |
| | 55° C. | SFRS9 | 31.655 | 0.101 |
| C157A/T467N | 55° C. | HPRT | 32.164 | 0.092 |
| | 55° C. | SFRS9 | 31.859 | 0.081 |
| C157A/G490A | 55° C. | HPRT | 31.540 | 0.140 |
| | 55° C. | SFRS9 | 31.058 | 0.072 |
| C157A/A502STOP | 55° C. | HPRT | 32.747 | 0.115 |
| | 55° C. | SFRS9 | 31.157 | 0.074 |
| C157A/L528Y | 55° C. | HPRT | 32.042 | 0.099 |
| | 55° C. | SFRS9 | 31.701 | 0.042 |
| C157A/R629M | 55° C. | HPRT | 31.641 | 0.050 |
| | 55° C. | SFRS9 | 31.190 | 0.099 |
| C157A/A646V | 55° C. | HPRT | 31.747 | 0.164 |
| | 55° C. | SFRS9 | 31.084 | 0.083 |
| R9A/R159P | 55° C. | HPRT | 31.684 | 0.199 |
| | 55° C. | SFRS9 | 31.025 | 0.029 |
| R9A/R173E | 55° C. | HPRT | 31.454 | 0.246 |
| | 55° C. | SFRS9 | 31.042 | 0.031 |
| R9A/T287D | 55° C. | HPRT | 30.783 | 0.082 |
| | 55° C. | SFRS9 | 30.181 | 0.091 |
| R9A/L333E | 55° C. | HPRT | 31.161 | 0.021 |
| | 55° C. | SFRS9 | 30.674 | 0.065 |
| R9A/V433T | 55° C. | HPRT | 30.871 | 0.060 |
| | 55° C. | SFRS9 | 30.193 | 0.034 |
| R9A/K435H | 55° C. | HPRT | 31.200 | 0.048 |
| | 55° C. | SFRS9 | 30.699 | 0.174 |
| R9A/T467N | 55° C. | HPRT | 32.134 | 0.229 |
| | 55° C. | SFRS9 | 31.731 | 0.027 |
| R9A/G490A | 55° C. | HPRT | 31.735 | 0.109 |
| | 55° C. | SFRS9 | 31.159 | 0.034 |
| R9A/A502STOP | 55° C. | HPRT | 33.524 | 0.032 |
| | 55° C. | SFRS9 | 31.459 | 0.046 |
| R9A/L528Y | 55° C. | HPRT | 31.918 | 0.053 |
| | 55° C. | SFRS9 | 31.608 | 0.125 |
| R9A/R629M | 55° C. | HPRT | 31.722 | 0.013 |
| | 55° C. | SFRS9 | 31.160 | 0.201 |
| R9A/A646V | 55° C. | HPRT | 32.096 | 0.246 |
| | 55° C. | SFRS9 | 31.705 | 0.176 |
| R159P/R173E | 55° C. | HPRT | 34.321 | 3.157 |
| | 55° C. | SFRS9 | 31.831 | 0.534 |
| R159P/V433T | 55° C. | HPRT | 30.944 | 0.081 |
| | 55° C. | SFRS9 | 30.426 | 0.127 |
| R159P/T467N | 55° C. | HPRT | 31.967 | 0.049 |
| | 55° C. | SFRS9 | 31.457 | 0.100 |
| R159P/A502STOP | 55° C. | HPRT | 31.828 | 0.154 |
| | 55° C. | SFRS9 | 30.809 | 0.091 |
| R159P/L528Y | 55° C. | HPRT | 31.784 | 0.085 |
| | 55° C. | SFRS9 | 31.513 | 0.084 |
| R159S/R173E | 55° C. | HPRT | 32.702 | 0.092 |
| | 55° C. | SFRS9 | 32.223 | 0.240 |
| R159S/V433T | 55° C. | HPRT | 31.565 | 0.138 |
| | 55° C. | SFRS9 | 31.063 | 0.092 |
| R159S/T467N | 55° C. | HPRT | 32.831 | 0.061 |
| | 55° C. | SFRS9 | 32.791 | 0.092 |
| R159S/A502STOP | 55° C. | HPRT | 33.145 | 0.146 |
| | 55° C. | SFRS9 | 31.523 | 0.039 |
| R159S/L528Y | 55° C. | HPRT | 32.606 | 0.286 |
| | 55° C. | SFRS9 | 31.716 | 0.069 |
| R173E/T287D | 55° C. | HPRT | 31.204 | 0.037 |
| | 55° C. | SFRS9 | 30.783 | 0.088 |
| R173E/L333E | 55° C. | HPRT | 31.158 | 0.038 |
| | 55° C. | SFRS9 | 30.714 | 0.033 |
| R173E/V433T | 55° C. | HPRT | 31.572 | 0.028 |
| | 55° C. | SFRS9 | 31.120 | 0.013 |
| R173E/K435H | 55° C. | HPRT | 31.391 | 0.103 |
| | 55° C. | SFRS9 | 31.002 | 0.055 |
| R173E/T467N | 55° C. | HPRT | 31.814 | 0.146 |
| | 55° C. | SFRS9 | 31.552 | 0.142 |
| R173E/G490A | 55° C. | HPRT | 32.349 | 0.128 |
| | 55° C. | SFRS9 | 32.027 | 0.089 |
| R173E/A502STOP | 55° C. | HPRT | 36.682 | 0.336 |
| | 55° C. | SFRS9 | 32.206 | 0.043 |
| R173E/L528Y | 55° C. | HPRT | 32.000 | 0.018 |
| | 55° C. | SFRS9 | 31.729 | 0.069 |
| R173E/R629M | 55° C. | HPRT | 32.299 | 0.165 |
| | 55° C. | SFRS9 | 32.096 | 0.065 |
| R173E/A646V | 55° C. | HPRT | 31.983 | 0.165 |
| | 55° C. | SFRS9 | 31.535 | 0.044 |
| T287D/V433T | 55° C. | HPRT | 30.888 | 0.054 |
| | 55° C. | SFRS9 | 30.277 | 0.027 |
| T287D/T467N | 55° C. | HPRT | 31.596 | 0.086 |
| | 55° C. | SFRS9 | 31.004 | 0.062 |
| T287D/A502STOP | 55° C. | HPRT | 31.292 | 0.084 |
| | 55° C. | SFRS9 | 30.458 | 0.090 |
| T287D/L528Y | 55° C. | HPRT | 31.416 | 0.070 |
| | 55° C. | SFRS9 | 30.984 | 0.069 |
| L333E/V433T | 55° C. | HPRT | 31.432 | 0.090 |
| | 55° C. | SFRS9 | 30.609 | 0.046 |
| L333E/T467N | 55° C. | HPRT | 31.700 | 0.093 |
| | 55° C. | SFRS9 | 31.195 | 0.085 |
| L333E/A502STOP | 55° C. | HPRT | 32.161 | 0.110 |
| | 55° C. | SFRS9 | 31.556 | 0.090 |
| L333E/L528Y | 55° C. | HPRT | 31.505 | 0.082 |
| | 55° C. | SFRS9 | 31.003 | 0.055 |
| V433T/K435H | 55° C. | HPRT | 31.596 | 0.056 |
| | 55° C. | SFRS9 | 30.923 | 0.177 |
| V433T/T467N | 55° C. | HPRT | 31.932 | 0.142 |
| | 55° C. | SFRS9 | 31.390 | 0.042 |
| V433T/G490A | 55° C. | HPRT | 31.963 | 0.139 |
| | 55° C. | SFRS9 | 31.401 | 0.111 |
| V433T/A502STOP | 55° C. | HPRT | 30.554 | 0.093 |
| | 55° C. | SFRS9 | 30.180 | 0.026 |
| V433T/L528Y | 55° C. | HPRT | 31.824 | 0.022 |
| | 55° C. | SFRS9 | 31.342 | 0.061 |
| V433T/R629M | 55° C. | HPRT | 31.982 | 0.152 |
| | 55° C. | SFRS9 | 31.530 | 0.067 |
| V433T/A646V | 55° C. | HPRT | 31.683 | 0.159 |
| | 55° C. | SFRS9 | 31.087 | 0.089 |
| K435H/T467N | 55° C. | HPRT | 32.041 | 0.193 |
| | 55° C. | SFRS9 | 31.687 | 0.037 |
| K435H/A502STOP | 55° C. | HPRT | 31.067 | 0.119 |
| | 55° C. | SFRS9 | 30.603 | 0.109 |
| K435H/L528Y | 55° C. | HPRT | 31.786 | 0.109 |
| | 55° C. | SFRS9 | 31.303 | 0.080 |
| T467N/G490A | 55° C. | HPRT | 32.283 | 0.350 |
| | 55° C. | SFRS9 | 31.646 | 0.075 |
| T467N/A502STOP | 55° C. | HPRT | 33.185 | 0.093 |
| | 55° C. | SFRS9 | 31.663 | 0.075 |
| T467N/L528Y | 55° C. | HPRT | 31.710 | 0.105 |
| | 55° C. | SFRS9 | 31.073 | 0.042 |
| T467N/R629M | 55° C. | HPRT | 32.429 | 0.026 |
| | 55° C. | SFRS9 | 32.116 | 0.164 |
| T467N/A646V | 55° C. | HPRT | 32.280 | 0.187 |
| | 55° C. | SFRS9 | 31.915 | 0.062 |
| G490A/A502STOP | 55° C. | HPRT | 33.617 | 0.079 |
| | 55° C. | SFRS9 | 31.761 | 0.119 |
| G490A/L528Y | 55° C. | HPRT | 32.042 | 0.158 |
| | 55° C. | SFRS9 | 31.573 | 0.086 |
| L528Y/R629M | 55° C. | HPRT | 31.797 | 0.038 |
| | 55° C. | SFRS9 | 31.204 | 0.052 |
| L528Y/A646V | 55° C. | HPRT | 32.406 | 0.163 |
| | 55° C. | SFRS9 | 32.010 | 0.042 |
| R9A/T287D/L333E | 55° C. | HPRT | 31.091 | 0.022 |
| | 55° C. | SFRS9 | 30.377 | 0.061 |
| C157A/T287D/L333E | 55° C. | HPRT | 30.916 | 0.019 |
| | 55° C. | SFRS9 | 30.381 | 0.085 |
| R173E/T287D/L333E | 55° C. | HPRT | 31.156 | 0.074 |
| | 55° C. | SFRS9 | 30.661 | 0.063 |
| T287D/L333E/V433T | 55° C. | HPRT | 30.960 | 0.079 |
| | 55° C. | SFRS9 | 30.195 | 0.027 |
| T287D/L333E/T467N | 55° C. | HPRT | 31.038 | 0.099 |
| | 55° C. | SFRS9 | 30.467 | 0.098 |
| T287D/L333E/A502STOP | 55° C. | HPRT | 30.653 | 0.119 |
| | 55° C. | SFRS9 | 30.208 | 0.089 |
| T287D/L333E/L528Y | 55° C. | HPRT | 30.950 | 0.041 |
| | 55° C. | SFRS9 | 30.217 | 0.060 |
| Mutant from App | 55° C. | HPRT | 32.370 | 0.060 |
| #20230031558 | 55° C. | SFRS9 | 32.141 | 0.057 |
| MMLV-II | 55° C. | HPRT | 38.902 | 1.147 |
| | 55° C. | SFRS9 | 38.411 | 1.168 |

TABLE 19
Two-Step cDNA synthesis by single, double, and triple
mutants of modified MMLV RTase. Data was generated
via qPCR assay for HPRT OR SFRS9, as indicated by the
“Target Name” column. Data is reported as Ct values.
Reverse Transcriptase Concentration = 5.2 nM final

| | cDNA | Target | Ct | Ct |
|---|---|---|---|---|
| MMLV RTase Variant | Temp | Name | Mean | SD |
| A500Q | 60° C. | HPRT | 34.143 | 0.391 |
| | 60° C. | HPRT | 33.469 | 0.189 |
| A619R | 60° C. | HPRT | 33.142 | 0.090 |
| | 60° C. | HPRT | 33.664 | 0.267 |
| R629T | 60° C. | HPRT | 33.406 | 0.441 |
| | 60° C. | HPRT | 34.918 | 0.404 |
| R9A/C157A | 60° C. | HPRT | 32.331 | 0.074 |
| | 60° C. | HPRT | 31.068 | 0.229 |
| C157A/R159P | 60° C. | HPRT | 32.079 | 0.186 |
| | 60° C. | HPRT | 31.838 | 0.124 |
| C157A/R159S | 60° C. | HPRT | 32.735 | 0.112 |
| | 60° C. | HPRT | 32.648 | 0.187 |
| C157A/R173E | 60° C. | HPRT | 31.858 | 0.102 |
| | 60° C. | HPRT | 35.107 | 0.359 |
| C157A/T287D | 60° C. | HPRT | 32.606 | 0.151 |
| | 60° C. | HPRT | 32.208 | 0.075 |
| C157A/L333E | 60° C. | HPRT | 34.780 | 3.694 |
| | 60° C. | HPRT | 31.496 | 0.087 |
| C157A/V433T | 60° C. | HPRT | 31.917 | 0.148 |
| | 60° C. | HPRT | 31.215 | 0.161 |
| C157A/K435H | 60° C. | HPRT | 31.734 | 0.119 |
| | 60° C. | HPRT | 31.183 | 0.175 |
| C157A/T467N | 60° C. | HPRT | 31.893 | 0.144 |
| | 60° C. | HPRT | 33.146 | 0.157 |
| C157A/G490A | 60° C. | HPRT | 32.005 | 0.131 |
| | 60° C. | HPRT | 36.234 | 0.728 |
| C157A/A502STOP | 60° C. | HPRT | 32.570 | 0.098 |
| | 60° C. | HPRT | 32.423 | 0.140 |
| C157A/L528Y | 60° C. | HPRT | 32.898 | 0.166 |
| | 60° C. | HPRT | 32.160 | 0.372 |
| C157A/R629M | 60° C. | HPRT | 31.042 | 0.098 |
| | 60° C. | HPRT | 32.221 | 0.045 |
| C157A/A646V | 60° C. | HPRT | 33.969 | 0.452 |
| | 60° C. | HPRT | 32.417 | 0.119 |
| R9A/R159P | 60° C. | HPRT | 33.264 | 0.230 |
| | 60° C. | HPRT | 32.062 | 0.056 |
| R9A/R173E | 60° C. | HPRT | 33.849 | 0.324 |
| | 60° C. | HPRT | 35.072 | 0.628 |
| R9A/T287D | 60° C. | HPRT | 32.574 | 0.068 |
| | 60° C. | HPRT | 31.919 | 0.105 |
| R9A/L333E | 60° C. | HPRT | 32.046 | 0.157 |
| | 60° C. | HPRT | 32.054 | 0.142 |
| R9A/V433T | 60° C. | HPRT | 32.351 | 0.286 |
| | 60° C. | HPRT | 33.152 | 0.190 |
| R9A/K435H | 60° C. | HPRT | 33.022 | 0.435 |
| | 60° C. | HPRT | 38.516 | 1.050 |
| R9A/T467N | 60° C. | HPRT | 33.051 | 0.216 |
| | 60° C. | HPRT | 33.482 | 0.199 |
| R9A/G490A | 60° C. | HPRT | 32.772 | 0.074 |
| | 60° C. | HPRT | 30.855 | 0.159 |
| R9A/A502STOP | 60° C. | HPRT | 31.778 | 0.122 |
| | 60° C. | HPRT | 34.453 | 0.450 |
| R9A/L528Y | 60° C. | HPRT | 32.105 | 0.062 |
| | 60° C. | HPRT | 31.506 | 0.062 |
| R9A/R629M | 60° C. | HPRT | 32.415 | 0.092 |
| | 60° C. | HPRT | 38.455 | 0.656 |
| R9A/A646V | 60° C. | HPRT | 32.149 | 0.171 |
| | 60° C. | HPRT | 31.617 | 0.246 |
| R159P/R173E | 60° C. | HPRT | 32.523 | 0.179 |
| | 60° C. | HPRT | 32.404 | 0.012 |
| R159P/V433T | 60° C. | HPRT | 34.646 | 0.412 |
| | 60° C. | HPRT | 32.453 | 0.255 |
| R159P/T467N | 60° C. | HPRT | 32.522 | 0.268 |
| | 60° C. | HPRT | 32.356 | 0.094 |
| R159P/A502STOP | 60° C. | HPRT | 33.316 | 0.085 |
| | 60° C. | HPRT | 33.433 | 0.519 |
| R159P/L528Y | 60° C. | HPRT | 32.458 | 0.256 |
| | 60° C. | HPRT | 32.796 | 0.100 |
| R159S/R173E | 60° C. | HPRT | 36.798 | 1.301 |
| | 60° C. | HPRT | 32.210 | 0.177 |
| R159S/V433T | 60° C. | HPRT | 33.492 | 0.286 |
| | 60° C. | HPRT | 33.197 | 0.082 |
| R159S/T467N | 60° C. | HPRT | 36.627 | 0.594 |
| | 60° C. | HPRT | 32.682 | 0.419 |
| R159S/A502STOP | 60° C. | HPRT | 32.435 | 0.227 |
| | 60° C. | HPRT | 33.346 | 0.328 |
| R159S/L528Y | 60° C. | HPRT | 31.243 | 0.153 |
| | 60° C. | HPRT | 31.512 | 0.300 |
| R173E/T287D | 60° C. | HPRT | 31.781 | 0.078 |
| | 60° C. | HPRT | 30.760 | 0.082 |
| R173E/L333E | 60° C. | HPRT | 31.088 | 0.102 |
| | 60° C. | HPRT | 34.111 | 0.257 |
| R173E/V433T | 60° C. | HPRT | 31.008 | 0.221 |
| | 60° C. | HPRT | 33.005 | 0.188 |
| R173E/K435H | 60° C. | HPRT | 39.791 | 0.296 |
| | 60° C. | SFRS9 | 33.151 | 0.322 |
| R173E/T467N | 60° C. | SFRS9 | 32.728 | 0.260 |
| | 60° C. | SFRS9 | 32.367 | 0.201 |
| R173E/G490A | 60° C. | SFRS9 | 32.933 | 0.047 |
| | 60° C. | SFRS9 | 32.657 | 0.117 |
| R173E/A502STOP | 60° C. | SFRS9 | 34.393 | 0.116 |
| | 60° C. | SFRS9 | 31.540 | 0.071 |
| R173E/L528Y | 60° C. | SFRS9 | 30.399 | 0.063 |
| | 60° C. | SFRS9 | 31.359 | 0.090 |
| R173E/R629M | 60° C. | SFRS9 | 31.133 | 0.072 |
| | 60° C. | SFRS9 | 31.971 | 0.101 |
| R173E/A646V | 60° C. | SFRS9 | 32.125 | 0.203 |
| | 60° C. | SFRS9 | 31.045 | 0.082 |
| T287D/V433T | 60° C. | SFRS9 | 31.796 | 0.087 |
| | 60° C. | SFRS9 | 31.838 | 0.117 |
| T287D/T467N | 60° C. | SFRS9 | 31.337 | 0.097 |
| | 60° C. | SFRS9 | 34.251 | 4.067 |
| T287D/A502STOP | 60° C. | SFRS9 | 31.008 | 0.057 |
| | 60° C. | SFRS9 | 31.146 | 0.109 |
| T287D/L528Y | 60° C. | SFRS9 | 30.439 | 0.092 |
| | 60° C. | SFRS9 | 31.257 | 0.189 |
| L333E/V433T | 60° C. | SFRS9 | 30.383 | 0.108 |
| | 60° C. | SFRS9 | 31.058 | 0.098 |
| L333E/T467N | 60° C. | SFRS9 | 32.129 | 0.111 |
| | 60° C. | SFRS9 | 31.279 | 0.086 |
| L333E/A502STOP | 60° C. | SFRS9 | 32.382 | 0.126 |
| | 60° C. | SFRS9 | 31.874 | 0.054 |
| L333E/L528Y | 60° C. | SFRS9 | 31.566 | 0.054 |
| | 60° C. | SFRS9 | 32.019 | 0.154 |
| V433T/K435H | 60° C. | SFRS9 | 31.413 | 0.053 |
| | 60° C. | SFRS9 | 30.466 | 0.029 |
| V433T/T467N | 60° C. | SFRS9 | 31.699 | 0.177 |
| | 60° C. | SFRS9 | 31.397 | 0.062 |
| V433T/G490A | 60° C. | SFRS9 | 31.795 | 0.079 |
| | 60° C. | SFRS9 | 32.366 | 0.164 |
| V433T/A502STOP | 60° C. | SFRS9 | 31.610 | 0.252 |
| | 60° C. | SFRS9 | 33.089 | 0.132 |
| V433T/L528Y | 60° C. | SFRS9 | 32.062 | 0.189 |
| | 60° C. | SFRS9 | 32.072 | 0.061 |
| V433T/R629M | 60° C. | SFRS9 | 31.212 | 0.015 |
| | 60° C. | SFRS9 | 31.302 | 0.024 |
| V433T/A646V | 60° C. | SFRS9 | 31.730 | 0.014 |
| | 60° C. | SFRS9 | 31.442 | 0.134 |
| K435H/T467N | 60° C. | SFRS9 | 32.229 | 0.083 |
| | 60° C. | SFRS9 | 32.377 | 0.276 |
| K435H/A502STOP | 60° C. | SFRS9 | 32.961 | 0.338 |
| | 60° C. | SFRS9 | 32.187 | 0.081 |
| K435H/L528Y | 60° C. | SFRS9 | 32.597 | 0.087 |
| | 60° C. | SFRS9 | 31.956 | 0.076 |
| T467N/G490A | 60° C. | SFRS9 | 30.214 | 0.101 |
| | 60° C. | SFRS9 | 31.379 | 0.121 |
| T467N/A502STOP | 60° C. | SFRS9 | 31.499 | 0.130 |
| | 60° C. | SFRS9 | 31.373 | 0.082 |
| T467N/L528Y | 60° C. | SFRS9 | 31.009 | 0.141 |
| | 60° C. | SFRS9 | 31.861 | 0.138 |
| T467N/R629M | 60° C. | SFRS9 | 33.243 | 0.138 |
| | 60° C. | SFRS9 | 31.446 | 0.066 |
| T467N/A646V | 60° C. | SFRS9 | 31.149 | 0.130 |
| | 60° C. | SFRS9 | 31.923 | 0.132 |
| G490A/A502STOP | 60° C. | SFRS9 | 31.864 | 0.092 |
| | 60° C. | SFRS9 | 31.585 | 0.091 |
| G490A/L528Y | 60° C. | SFRS9 | 31.938 | 0.102 |
| | 60° C. | SFRS9 | 32.145 | 0.250 |
| L528Y/R629M | 60° C. | SFRS9 | 31.497 | 0.069 |
| | 60° C. | SFRS9 | 32.506 | 0.078 |
| L528Y/A646V | 60° C. | SFRS9 | 31.769 | 0.223 |
| | 60° C. | SFRS9 | 31.997 | 0.116 |
| R9A/T287D/L333E | 60° C. | SFRS9 | 31.790 | 0.195 |
| | 60° C. | SFRS9 | 32.775 | 0.291 |
| C157A/T287D/L333E | 60° C. | SFRS9 | 31.470 | 0.017 |
| | 60° C. | SFRS9 | 32.916 | 0.173 |
| R173E/T287D/L333E | 60° C. | SFRS9 | 32.544 | 0.303 |
| | 60° C. | SFRS9 | 33.007 | 0.263 |
| T287D/L333E/V433T | 60° C. | SFRS9 | 32.071 | 0.192 |
| | 60° C. | SFRS9 | 31.802 | 0.010 |
| T287D/L333E/T467N | 60° C. | SFRS9 | 32.804 | 0.102 |
| | 60° C. | SFRS9 | 30.924 | 0.103 |
| T287D/L333E/A502STOP | 60° C. | SFRS9 | 31.205 | 0.632 |
| | 60° C. | SFRS9 | 31.232 | 0.092 |
| T287D/L333E/L528Y | 60° C. | SFRS9 | 30.450 | 0.071 |
| | 60° C. | SFRS9 | 30.685 | 0.073 |
| Mutant from App | 60° C. | SFRS9 | 31.461 | 0.150 |
| #20230031558 | 60° C. | SFRS9 | 30.458 | 0.072 |
| MMLV-II | 60° C. | SFRS9 | 32.254 | 0.134 |
| | 60° C. | SFRS9 | 37.873 | 0.741 |

TABLE 20
Two-Step cDNA synthesis by single, double, and triple
mutants of modified MMLV RTase. Data was generated
via qPCR assay for HPRT OR SFRS9, as indicated by the
“Target Name” column. Data is reported as Ct values.
Reverse Transcriptase Concentration = 5.2 nM final

| | cDNA | Target | Ct | Ct |
|---|---|---|---|---|
| RTase | Temp | Name | Mean | SD |
| A500Q | 65° C. | HPRT | 35.278 | 0.102 |
| | 65° C. | HPRT | 34.512 | 0.299 |
| A619R | 65° C. | HPRT | 34.580 | 0.264 |
| | 65° C. | HPRT | 34.732 | 0.301 |
| R629T | 65° C. | HPRT | 34.044 | 0.347 |
| | 65° C. | HPRT | 35.241 | 0.571 |
| R9A/C157A | 65° C. | HPRT | 33.567 | 0.329 |
| | 65° C. | HPRT | 31.749 | 0.093 |
| C157A/R159P | 65° C. | HPRT | 32.819 | 0.263 |
| | 65° C. | HPRT | 32.835 | 0.160 |
| C157A/R159S | 65° C. | HPRT | 33.787 | 0.404 |
| | 65° C. | HPRT | 33.794 | 0.268 |
| C157A/R173E | 65° C. | HPRT | 32.571 | 0.061 |
| | 65° C. | HPRT | 36.043 | 0.609 |
| C157A/T287D | 65° C. | HPRT | 33.229 | 0.251 |
| | 65° C. | HPRT | 32.799 | 0.381 |
| C157A/L333E | 65° C. | HPRT | 32.468 | 0.143 |
| | 65° C. | HPRT | 32.248 | 0.192 |
| C157A/V433T | 65° C. | HPRT | 33.168 | 0.432 |
| | 65° C. | HPRT | 31.735 | 0.119 |
| C157A/K435H | 65° C. | HPRT | 32.821 | 0.210 |
| | 65° C. | HPRT | 32.221 | 0.117 |
| C157A/T467N | 65° C. | HPRT | 33.185 | 0.313 |
| | 65° C. | HPRT | 34.016 | 0.342 |
| C157A/G490A | 65° C. | HPRT | 32.846 | 0.307 |
| | 65° C. | HPRT | 38.052 | 0.763 |
| C157A/A502STOP | 65° C. | HPRT | 33.440 | 0.256 |
| | 65° C. | HPRT | 33.422 | 0.134 |
| C157A/L528Y | 65° C. | HPRT | 34.013 | 0.365 |
| | 65° C. | HPRT | 33.366 | 0.360 |
| C157A/R629M | 65° C. | HPRT | 31.866 | 0.082 |
| | 65° C. | HPRT | 32.889 | 0.299 |
| C157A/A646V | 65° C. | HPRT | 34.979 | 0.686 |
| | 65° C. | HPRT | 32.894 | 0.323 |
| R9A/R159P | 65° C. | HPRT | 33.964 | 0.309 |
| | 65° C. | HPRT | 33.127 | 0.410 |
| R9A/R173E | 65° C. | HPRT | 34.704 | 0.563 |
| | 65° C. | HPRT | 35.448 | 0.446 |
| R9A/T287D | 65° C. | HPRT | 33.694 | 0.100 |
| | 65° C. | HPRT | 32.840 | 0.068 |
| R9A/L333E | 65° C. | HPRT | 33.318 | 0.020 |
| | 65° C. | HPRT | 34.142 | 0.481 |
| R9A/V433T | 65° C. | HPRT | 33.523 | 0.369 |
| | 65° C. | HPRT | 34.931 | 0.513 |
| R9A/K435H | 65° C. | HPRT | 33.997 | 0.256 |
| | 65° C. | HPRT | 38.781 | 0.863 |
| R9A/T467N | 65° C. | HPRT | 33.934 | 0.290 |
| | 65° C. | HPRT | 34.447 | 0.309 |
| R9A/G490A | 65° C. | HPRT | 33.937 | 0.234 |
| | 65° C. | HPRT | 31.924 | 0.248 |
| R9A/A502STOP | 65° C. | HPRT | 33.040 | 0.368 |
| | 65° C. | HPRT | 35.997 | 0.496 |
| R9A/L528Y | 65° C. | HPRT | 32.895 | 0.252 |
| | 65° C. | HPRT | 32.995 | 0.247 |
| R9A/R629M | 65° C. | HPRT | 33.910 | 0.220 |
| | 65° C. | HPRT | 39.282 | 1.015 |
| R9A/A646V | 65° C. | HPRT | 32.574 | 0.044 |
| | 65° C. | HPRT | 33.455 | 0.270 |
| R159P/R173E | 65° C. | HPRT | 34.179 | 0.284 |
| | 65° C. | HPRT | 33.328 | 0.352 |
| R159P/V433T | 65° C. | HPRT | 35.488 | 0.413 |
| | 65° C. | HPRT | 34.089 | 0.174 |
| R159P/T467N | 65° C. | HPRT | 34.212 | 0.269 |
| | 65° C. | HPRT | 33.629 | 0.500 |
| R159P/A502STOP | 65° C. | HPRT | 35.403 | 0.196 |
| | 65° C. | HPRT | 35.082 | 0.599 |
| R159P/L528Y | 65° C. | HPRT | 34.092 | 0.186 |
| | 65° C. | HPRT | 33.526 | 0.295 |
| R159S/R173E | 65° C. | HPRT | 37.816 | 0.520 |
| | 65° C. | HPRT | 33.492 | 0.110 |
| R159S/V433T | 65° C. | HPRT | 34.818 | 0.448 |
| | 65° C. | HPRT | 34.947 | 0.507 |
| R159S/T467N | 65° C. | HPRT | 37.406 | 1.851 |
| | 65° C. | HPRT | 33.635 | 0.452 |
| R159S/A502STOP | 65° C. | HPRT | 33.308 | 0.104 |
| | 65° C. | HPRT | 34.114 | 0.644 |
| R159S/L528Y | 65° C. | HPRT | 32.134 | 0.147 |
| | 65° C. | HPRT | 31.762 | 0.080 |
| R173E/T287D | 65° C. | HPRT | 32.678 | 0.237 |
| | 65° C. | HPRT | 31.670 | 0.034 |
| R173E/L333E | 65° C. | HPRT | 32.062 | 0.100 |
| | 65° C. | HPRT | 35.932 | 0.531 |
| R173E/V433T | 65° C. | HPRT | 31.804 | 0.219 |
| | 65° C. | HPRT | 33.965 | 0.358 |
| R173E/K435H | 65° C. | HPRT | 38.723 | 0.933 |
| | 65° C. | SFRS9 | 34.641 | 0.366 |
| R173E/T467N | 65° C. | SFRS9 | 33.704 | 0.008 |
| | 65° C. | SFRS9 | 33.249 | 0.190 |
| R173E/G490A | 65° C. | SFRS9 | 33.527 | 0.211 |
| | 65° C. | SFRS9 | 33.270 | 0.254 |
| R173E/A502STOP | 65° C. | SFRS9 | 34.418 | 0.427 |
| | 65° C. | SFRS9 | 32.399 | 0.069 |
| R173E/L528Y | 65° C. | SFRS9 | 30.936 | 0.115 |
| | 65° C. | SFRS9 | 31.931 | 0.086 |
| R173E/R629M | 65° C. | SFRS9 | 31.807 | 0.176 |
| | 65° C. | SFRS9 | 32.739 | 0.038 |
| R173E/A646V | 65° C. | SFRS9 | 32.652 | 0.261 |
| | 65° C. | SFRS9 | 31.715 | 0.099 |
| T287D/V433T | 65° C. | SFRS9 | 32.296 | 0.132 |
| | 65° C. | SFRS9 | 32.383 | 0.109 |
| T287D/T467N | 65° C. | SFRS9 | 31.700 | 0.017 |
| | 65° C. | SFRS9 | 31.757 | 0.124 |
| T287D/A502STOP | 65° C. | SFRS9 | 31.351 | 0.091 |
| | 65° C. | SFRS9 | 31.672 | 0.136 |
| T287D/L528Y | 65° C. | SFRS9 | 30.920 | 0.035 |
| | 65° C. | SFRS9 | 31.833 | 0.100 |
| L333E/V433T | 65° C. | SFRS9 | 31.014 | 0.333 |
| | 65° C. | SFRS9 | 31.751 | 0.143 |
| L333E/T467N | 65° C. | SFRS9 | 32.761 | 0.082 |
| | 65° C. | SFRS9 | 31.562 | 0.092 |
| L333E/A502STOP | 65° C. | SFRS9 | 32.503 | 0.223 |
| | 65° C. | SFRS9 | 32.412 | 0.009 |
| L333E/L528Y | 65° C. | SFRS9 | 32.104 | 0.106 |
| | 65° C. | SFRS9 | 32.806 | 0.129 |
| V433T/K435H | 65° C. | SFRS9 | 32.162 | 0.130 |
| | 65° C. | SFRS9 | 30.988 | 0.090 |
| V433T/T467N | 65° C. | SFRS9 | 32.288 | 0.233 |
| | 65° C. | SFRS9 | 31.552 | 0.213 |
| V433T/G490A | 65° C. | SFRS9 | 31.812 | 0.109 |
| | 65° C. | SFRS9 | 32.592 | 0.076 |
| V433T/A502STOP | 65° C. | SFRS9 | 31.794 | 0.128 |
| | 65° C. | SFRS9 | 33.810 | 0.355 |
| V433T/L528Y | 65° C. | SFRS9 | 32.099 | 0.130 |
| | 65° C. | SFRS9 | 32.736 | 0.222 |
| V433T/R629M | 65° C. | SFRS9 | 31.497 | 0.040 |
| | 65° C. | SFRS9 | 31.947 | 0.063 |
| V433T/A646V | 65° C. | SFRS9 | 32.352 | 0.180 |
| | 65° C. | SFRS9 | 31.771 | 0.115 |
| K435H/T467N | 65° C. | SFRS9 | 32.711 | 0.252 |
| | 65° C. | SFRS9 | 32.828 | 0.157 |
| K435H/A502STOP | 65° C. | SFRS9 | 33.859 | 0.338 |
| | 65° C. | SFRS9 | 32.584 | 0.051 |
| K435H/L528Y | 65° C. | SFRS9 | 32.986 | 0.114 |
| | 65° C. | SFRS9 | 32.169 | 0.064 |
| T467N/G490A | 65° C. | SFRS9 | 30.805 | 0.050 |
| | 65° C. | SFRS9 | 31.732 | 0.077 |
| T467N/A502STOP | 65° C. | SFRS9 | 32.119 | 0.077 |
| | 65° C. | SFRS9 | 32.038 | 0.167 |
| T467N/L528Y | 65° C. | SFRS9 | 31.929 | 0.122 |
| | 65° C. | SFRS9 | 32.591 | 0.150 |
| T467N/R629M | 65° C. | SFRS9 | 33.980 | 0.437 |
| | 65° C. | SFRS9 | 32.022 | 0.046 |
| T467N/A646V | 65° C. | SFRS9 | 32.249 | 0.046 |
| | 65° C. | SFRS9 | 32.868 | 0.099 |
| G490A/A502STOP | 65° C. | SFRS9 | 32.451 | 0.114 |
| | 65° C. | SFRS9 | 32.032 | 0.217 |
| G490A/L528Y | 65° C. | SFRS9 | 32.763 | 0.084 |
| | 65° C. | SFRS9 | 33.005 | 0.189 |
| L528Y/R629M | 65° C. | SFRS9 | 32.110 | 0.056 |
| | 65° C. | SFRS9 | 33.757 | 0.311 |
| L528Y/A646V | 65° C. | SFRS9 | 32.849 | 0.107 |
| | 65° C. | SFRS9 | 32.978 | 0.198 |
| R9A/T287D/L333E | 65° C. | SFRS9 | 32.639 | 0.150 |
| | 65° C. | SFRS9 | 33.911 | 0.314 |
| C157A/T287D/L333E | 65° C. | SFRS9 | 32.340 | 0.144 |
| | 65° C. | SFRS9 | 33.546 | 0.235 |
| R173E/T287D/L333E | 65° C. | SFRS9 | 33.329 | 0.206 |
| | 65° C. | SFRS9 | 33.667 | 0.068 |
| T287D/L333E/V433T | 65° C. | SFRS9 | 32.527 | 0.132 |
| | 65° C. | SFRS9 | 32.268 | 0.088 |
| T287D/L333E/T467N | 65° C. | SFRS9 | 33.295 | 0.096 |
| | 65° C. | SFRS9 | 31.464 | 0.076 |
| T287D/L333E/A502STOP | 65° C. | SFRS9 | 31.076 | 0.088 |
| | 65° C. | SFRS9 | 31.584 | 0.126 |
| T287D/L333E/L528Y | 65° C. | SFRS9 | 31.091 | 0.163 |
| | 65° C. | SFRS9 | 31.021 | 0.125 |
| Mutant from App | 65° C. | SFRS9 | 32.321 | 0.111 |
| #20230031558 | 65° C. | SFRS9 | 31.027 | 0.141 |
| MMLV-II | 65° C. | SFRS9 | 33.175 | 0.202 |
| | 65° C. | SFRS9 | 37.701 | 0.725 |

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### DISCUSSION OF POSSIBLE EMBODIMENTS

The following are non-exclusive descriptions of possible embodiments of the present invention.

An embodiment of an isolated Moloney murine leukemia virus (MMLV) reverse transcriptase (RTase) mutant includes an amino acid sequence as set forth in any of SEQ ID NOs: 278-432.

An embodiment of a nucleotide includes a nucleotide encoding a foregoing isolated MMLV RTase mutant.

An embodiment of a cDNA includes a cDNA encoding a foregoing isolated MMLV RTase mutant.

An embodiment of a kit includes a kit comprising a foregoing isolated MMLV RTase mutant.

An embodiment of a composition includes a composition comprising a foregoing isolated MMLV RTase mutant.

A further embodiment of an isolated MMLV RTase variant includes a substitution at a position corresponding to least one of position 9, position 157, position 159, position 173, position 287, position 333, position 433, position 435, position 467, position 490, position 502, position 629, or position 646 of any of SEQ ID NOs: 275-277.

The isolated MMLV RTase variant of the preceding paragraph can optionally include, additionally and/or alternatively, any one or more of the following features, configurations and/or additional components:

A further embodiment of the forgoing isolated MMLV RTase variant, wherein the variant comprises one or more substitutions selected from a group consisting of R9A, R9L, C157A, R159P, R159S, R173E, T287D, L333E, L333S, V433T, K435H, T467N, G490A, G490N, A502STOP, R629M, A646V, T287D+L333E, C157A+T287D, R9A+T287D, R9A+V433T, T287D+V433T, T287D+A502STOP, V433T+A502STOP, T287D+L333E+V433T, T287D+L333E+A502STOP, and T287D+L333E+L528Y.

A further embodiment of the forgoing isolated MMLV RTase variant, wherein the variant comprises one or more substitutions selected from a group consisting of R9A, C157A, R173E, V433T, T467N, A502STOP, L528Y, T287D+L333E, C157A+T287D, R9A+T287D, R9A+V433T, T287D+V433T, T287D+A502STOP, V433T+A502STOP, T287D+L333E+V433T, T287D+L333E+A502STOP, and T287D+L333E+L528Y.

An embodiment of a nucleotide includes a nucleotide encoding a foregoing isolated MMLV RTase variant.

An embodiment of a cDNA includes a cDNA encoding a foregoing isolated MMLV RTase variant.

An embodiment of a kit includes a kit comprising a foregoing isolated MMLV RTase variant.

An embodiment of a composition includes a composition comprising a foregoing isolated MMLV RTase variant.

An embodiment of an isolated prime editor fusion protein includes a nuclease domain and a reverse transcriptase domain having a sequence of an MMLV RTase mutant of a foregoing embodiment.

A method of synthesizing copy deoxyribonucleic acid (cDNA) includes providing a ribonucleic acid template and contacting the ribonucleic acid template with an MMLV RTase mutant of a foregoing embodiment to synthesize cDNA.

A method of gene editing includes providing a sample including a double-stranded target DNA sequence; and contacting the double-stranded target DNA sequence with a prime editor, the prime editor comprising: first domain comprising a nucleic acid programmable DNA binding protein; a second domain comprising a MMLV RTase mutant of a foregoing embodiment; and a prime editing guide RNA.

The method of gene editing of the preceding paragraph can optionally include, additionally and/or alternatively, any one or more of the following features, configurations and/or additional components:

A further embodiment of the foregoing method of gene editing, wherein the nucleic acid programmable DNA binding protein comprises a nickase domain.

A method of performing reverse transcriptase-polymerase chain reaction (RT-PCR) including providing a ribonucleic acid template; contacting the ribonucleic acid template with an MMLV RTase variant of a foregoing embodiment to synthesize cDNA; and contacting the cDNA with a DNA polymerase to amplify the cDNA.

1. An isolated Moloney murine leukemia virus (MMLV) reverse transcriptase (RTase) mutant comprising an amino acid sequence as set forth in any of SEQ ID NOs: 278-432.

2. A nucleotide encoding the MMLV RTase mutant of claim 1.

3. A cDNA encoding the MMLV RTase mutant of claim 1.

4. A kit comprising the MMLV RTase mutant of claim 1.

5. A composition comprising the MMLV RTase mutant of claim 1.

6. An MMLV RTase variant comprising a substitution at a position corresponding to least one of position 9, position 157, position 159, position 173, position 287, position 333, position 433, position 435, position 467, position 490, position 502, position 629, or position 646 of any of SEQ ID NOs: 275-277.

7. The MMLV RTase variant of claim 6, wherein the variant comprises one or more substitutions selected from a group consisting of R9A, R9L, C157A, R159P, R159S, R173E, T287D, L333E, L333S, V433T, K435H, T467N, G490A, G490N, A502STOP, R629M, A646V, T287D+L333E, C157A+T287D, R9A+T287D, R9A+V433T, T287D+V433T, T287D+A502STOP, V433T+A502STOP, T287D+L333E+V433T, T287D+L333E+A502STOP, and T287D+L333E+L528Y.

8. The MMLV RTase variant of claim 6, wherein the variant comprises one or more substitutions selected from a group consisting of R9A, C157A, R173E, V433T, T467N, A502STOP, L528Y, T287D+L333E, C157A+T287D, R9A+T287D, R9A+V433T, T287D+V433T, T287D+A502STOP, V433T+A502STOP, T287D+L333E+V433T, T287D+L333E+A502STOP, and T287D+L333E+L528Y.

9. A nucleotide encoding the MMLV RTase mutant of claim 6.

10. A cDNA encoding the MMLV RTase mutant of claim 6.

11. A kit comprising the MMLV RTase mutant of claim 6.

12. A composition comprising the MMLV RTase mutant of claim 6.

13. An isolated prime editor fusion protein comprising:
a nuclease domain; and
a reverse transcriptase domain having a sequence of the MMLV RTase mutant of claim 6.

14. A method of synthesizing copy deoxyribonucleic acid (cDNA), the method comprising:
providing a ribonucleic acid template; and
contacting the ribonucleic acid template with an MMLV RTase variant of claim 6 to synthesize cDNA.

15. A method of gene editing, the method comprising:
providing a sample including a double-stranded target DNA sequence; and
contacting the double-stranded target DNA sequence with a prime editor, the prime editor comprising:
first domain comprising a nucleic acid programmable DNA binding protein;
a second domain comprising the MMLV RTase variant of claim 6; and
a prime editing guide RNA.

16. The method of claim 15, wherein the nucleic acid programmable DNA binding protein comprises a nickase domain.

17. A method of performing reverse transcriptase-polymerase chain reaction (RT-PCR), the method comprising:
providing a ribonucleic acid template;
contacting the ribonucleic acid template with an MMLV RTase variant of claim 6 to synthesize cDNA; and
contacting the cDNA with a DNA polymerase to amplify the cDNA.
